(19) United States
(12) Patent Application Publication
(10) Pub. No.: US 2013/0006342 A1
(43) Pub. Date: Jan. 3, 2013

(54) EXPRESSION SIGNATURE IN PERIPHERAL BLOOD FOR DETECTION OF AORTIC ANEURYSM

(75) Inventors: Yulei WANG, Foster City, CA (US); Catalin Barbacioru, Fremont, CA (US); Raymond R. Samaha, Soquel, CA (US); John A. Elefteriades, New Haven, CT (US)

(73) Assignee: Applied BioSystems, LLC, Carlsbad, CA (US)

(21) Appl. No.: 13/611,240

(22) Filed: Sep. 12, 2012

Related U.S. Application Data
(63) Continuation of application No. 11/981,143, filed on Oct. 30, 2007.
(60) Provisional application No. 60/855,954, filed on Oct. 31, 2006.
(60) Provisional application No. 60/856,491, filed on Nov. 2, 2006.

(51) Int. Cl. C40B 30/04; A61F 2/06; C12Q 1/68

(52) U.S. Cl. 623/1.1; 506/9; 435/6.12

(57) ABSTRACT

We hypothesized that gene expression patterns in peripheral blood cells may correlate with TAA disease status, and carried out a comprehensive gene expression survey on peripheral blood cells obtained from TAA patients and normal individuals. A distinct gene expression profile in peripheral blood cells can classify TAA patients from normal individuals. The genes provided by the present teachings define a set of diagnostic markers, thus providing a blood-based gene expression test to facilitate early detection of TAA disease. Methods of distinguishing ascending from descending TAA are also provided, as are methods of distinguishing familial from sporadic TAA.

### CROSS-REFERENCE TO RELATED APPLICATIONS

This application is a continuation of U.S. Ser. No. 11/981,143, filed Oct. 30, 2007, which claims priority to U.S. Ser. No. 60/856,491, filed Nov. 2, 2006, and U.S. Ser. No. 60/855,954, filed Oct. 31, 2006, the disclosures of which are herein incorporated by reference.

### FIELD

The present teachings relate generally to molecular biology, and in particular to methods for detecting and treating thoracic aortic aneurysm.

### INTRODUCTION

Thoracic aortic aneurysm (TAA), without surgical treatment, is a lethal disease. With elective surgical treatment, near-normal prognosis is restored. Thus, in aneurysm disease, the early diagnosis is the key to the treatment decelerating the progression of TAA and to the timely elective surgery. Because TAA is almost invariably asymptomatic until rupture or dissection occur, methods of detection need to be applied to asymptomatic individuals. Physical examinations are generally unable to detect thoracic aortic aneurysm, thus imaging technologies (echocardiography (ECHO), computerized tomography (CT), or magnetic resonance imaging (MRI)) are utilized to diagnose.

Thoracic aortic disease runs in families. Screening of family members by radiographic imaging modalities is just beginning to be performed, mainly at specialized aortic centers. While radiographic screening is extremely valuable, many patients who have increased genetic risk to develop aneurysms later in life may have no recognizable enlargement of the aorta at the time of screening, even with state-of-the-art imaging technologies. This is especially true for young offspring of affected individuals. For all these reasons, a rapid, standardized blood test capable of detecting individuals at risk for the aneurysm disease would represent a major advance in clinical care. However, in the case of TAA, it is difficult to obtain the affected tissue itself for analysis, so we look to peripheral blood as an easily accessible source of cells that may be used diagnostically as surrogates for direct sampling of diseased tissues. Circulating leukocytes serve as a vigilant and comprehensive surveillance of the body for signs of infection, inflammation, and other abnormality.

Peripheral blood cells have been used to identify gene expression signatures for autoimmune diseases such as systemic lupus erythematosus (SLE) (Mandel et al., Clin Exp Immunol 138, 164-70 (2004); Baechler, E. C. et al. Proc Natl Acad Sci USA 100, 2610-5 (2003)), rheumatoid arthritis (RA) (Batliwalla, F. M. et al. Genes Immun 6, 388-97 (2005)), and multiple sclerosis (MS) (Bomprezzi et al. Hum Mol Genet 12, 2191-9 (2003); Achiron et al. Clin Dev Immunol 11, 299-305 (2004); Achiron et al., Ann Neurol 55, 410-7 (2004)). These signatures genes have been also shown to be useful in identifying pathways relevant to disease and to predict response to therapy. Although the mechanisms responsible for the formation of TAA remain elusive, the importance of genetic predisposition (Elefteriades et al., J Am Coll Cardiol 39, 180-1 (2002); Guo, D. et al. Circulation 103, 2461-8 (2001); Hasham et al. Circulation 107, 3184-90 (2003); Khau Van Kien et al., Circulation 112, 200-6 (2005); SoRelle Circulation 107, e9055-6 (2003); Wung et al., J Cardiovasc Nurs 19, 409-16 (2004)), inflammation (Tang, et al. Faseb J 19, 1528-30 (2005); Koullias et al. J Thorac Cardiovasc Surg 130, 677 e1-2 (2005); Koullias et al., Ann Thorac Surg 78, 2106-10; discussion 2110-1 (2004), Walton et al. Circulation 100, 48-54 (1999)), and adaptive cellular immune responses (Davis et al. J Surg Res 101, 152-6 (2001); Ocana et al., Atherosclerosis 170, 39-48 (2003); Schonbeck et al., Am J Pathol 161, 499-506 (2002)) in the development of aneurysm disease has been well appreciated.

We thus hypothesized that gene expression patterns in peripheral blood cells may reflect TAA disease status. In the present teachings, we carried out a comprehensive gene expression survey on peripheral blood cells obtained from TAA patients and normal individuals, using the Applied Biosystems Human Genome Survey Microarray representing 29,098 individual genes. Identification of a distinct molecular RNA signature in peripheral blood provides a rapid diagnosis of the aneurysm diathesis by a bedside test. Such blood-based test could be made available in hospitals, laboratories, physician offices, and, especially, emergency rooms. A RNA aneurysm expression profile could also provide insights into the molecular pathogenesis of aneurysmal degeneration of the aortic wall.

### SUMMARY

In some embodiments, the present teachings provide a method of diagnosing a human subject with TAA, the method comprising; detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with TAA.

In some embodiments, the present teachings provide a method of distinguishing ascending thoracic aortic aneurysm from descending thoracic aortic aneurysm comprising; detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with an ascending aortic aneurysm, wherein the plurality of genes in the test sample are overexpressed in the ascending aortic aneurysm as compared with the control sample.

In some embodiments, the present teachings provide a method of distinguishing sporadic thoracic aortic aneurysm from familial thoracic aortic aneurysm comprising; detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the thoracic aortic aneurysm is sporadic, wherein the plurality of genes in the test sample are overexpressed in the test sample as compared with the control sample.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### DESCRIPTION OF THE FIGURES AND FILES

FIG. 1 Hierarchical clustering of 61 whole blood samples analyzed by Applied Biosystem Expression Arrays using the 1207 differentially expressed genes determined by SAM analysis. The level of expression of each gene in each sample, relative to the mean level of expression of that gene across all the samples, is represented using a redblack-green color scale as shown in the key (green: below mean; black: equal to mean; red: above mean). (A). Scaled down representation of the entire cluster of the 1207 signature genes and 61 whole blood samples. (B). Experimental dendrogram displaying the clustering of the samples into two main branches: the TAA branch (red) and the control branch (blue) with a few exceptions. (C). Gene expression pattern of representative genes within biological pathways that are statistically significantly overrepresented (random overlapping p-value <0.05) by the up-regulated (red bars) or the down-regulated (blue bars) signature genes of TAA.

FIG. 2 Two-dimensional cluster diagrams. (A). 144 signature genes characterizing the ascending and descending TAA subtypes; (B). 113 signature genes characterizing the TAA with or without family history. Representative genes associated with overrepresented molecular functions/biological processes/pathways are listed.

FIG. 3 A set of 41 classifier genes were identified via 10-fold cross-validation on the 61-sample training set. (A). Prediction accuracy, sensitivity and specificity of the 41 classifier genes, error bar represents ±1 Stdev among 100 times of independent 10-fold cross-validation process; (B). 3D Plots of the first three principal components based on PCA analysis. The segregation between TAA and control samples is evident with only a few exceptions.

FIG. 4 Validation of the prediction models by testing independent sample set analyzed by microarray. (A). Probability of being either TAA (case) or Normal (control) for each testing sample; (B). Contingency table depicting the predicted and actual class membership. (C). Predicting accuracy, sensitivity and specificity.

FIG. 5 Validation of the 41 classifier genes using TaqMan based real-time PCR. Expression profile of the 41 classifier genes was measured in each of the 82 samples by real-time PCR using TaqMan® Gene Expression Assays. Based on TaqMan data, the coefficient of the 41 classifier genes were re-learned from the 52 training samples and used to predict the 30 testing samples using the same method applied to microarray data. (A). Predicted probabilities of being TAA (case) and Normal (control) for each testing sample; (B). Contingency table depicting the predicted and actual class membership; (C). Predicting accuracy, sensitivity and specificity.

FIG. 6: Determination of the optimal set of classifier genes using 10-fold cross-validation on the training set (see detailed description in Methods). Prediction accuracy using different number of classifier genes was illustrated; the error bar indicates ±1 Stdev among 100 times of independent 10-fold cross-validation process.

FIG. 7: 144 candidate signature genes distinguishing Ascending vs. Descending TAA, identified based on microarray data and SAM analysis (ave. FC>1.3 and FDR<2%).

FIG. 8: 113 candidate signature genes distinguishing familial vs. sporadic TAA, identified based on microarray data and SAM analysis (ave. FC>1.3 and FDR<4%)

FIG. 9: List of the 41 classifier genes classify TAA from normal individuals.

The practice of the present invention may employ conventional techniques and descriptions of organic chemistry, polymer technology, molecular biology (including recombinant techniques), cell biology, biochemistry, and immunology, which are within the skill of the art. Such conventional techniques include oligonucleotide synthesis, hybridization, extension reaction, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

### Some Methods

The following methods sections are presented as illustrative and are not intended to limit the scope of the presently claimed invention. Additional approaches for determining expression profiles consistent with the presently claimed invention are known to one of ordinary skill. Such approaches can be found, for example, in U.S. Pat. No. 7,108,969, which is hereby incorporated by reference. The gene expression monitoring of the present teachings may comprise any of a variety of approaches, including a nucleic acid probe array (such as those described above), membrane blot (such as used in hybridization analysis such as Northern, Southern, dot, and the like), microwells, sample tubes, gels, beads or fibers (or any solid support comprising bound nucleic acids). See U.S. Pat. Nos. 5,770,722, 5,874,219, 5,744,305, 5,677,195 and 5,445,934, 5,800,992 which are expressly incorporated herein by reference in their entireties for all purposes. In some embodiments, the gene expression monitoring system can comprise PCR, for example real-time PCR such as TaqMan®.

### Blood Samples Collection

Peripheral blood was harvested from 58 TAA patients and 36 spousal controls using PAXgene™ tubes (Qiagen, Valencia, Calif.). All patients (39 male, 19 female) harbored known thoracic aortic aneurysms, based on radiographic images (ECHO, CT, or MRI) and/or operative findings. Patients with Marfan disease were specifically excluded. Spousal controls were chosen because of the similarities in age, ethnicity, geography, and diet that usually characterize husband and wife. Complete blood counts of all blood samples were carried out at the Clinical Laboratory of Yale-New Haven Hospital.

### RNA Preparation

The PAXgene™ tubes were frozen at the collection site and shipped on dry ice. After thawing at room temperature for at least 2 hours, total RNA was extracted from the approximately 2.5 ml of peripheral blood in each tube following the manufacturer's recommended protocol (Preanalytix Blood RNA Kit Handbook, Qiagen). The quality and integrity of the total RNA was evaluated on the 2100 Bioanalyzer (Agilent Technologies) and the concentration was measured using a NanoDrop spectrophotometer (NanoDrop Technologies).

### Applied Biosystems Expression Array Analysis

The Applied Biosystems Human Genome Survey Microarray v2.0 (P/N 4337467) contains 33,096 60-mer oligonucleotide probes representing 29,098 individual human genes. Digoxigenin-UTP labeled cRNA was generated and amplified from 1 μg of total RNA from each sample using Applied Biosystems Chemiluminescent RT-IVT Labeling Kit v 1.0 (P/N 4340472) according to the manufacturer's protocol (P/N 4339629). 20 μg Digoxigenin-UTP labeled cRNA was used for each hybridization, which was performed for 16 hrs at 55 C. Chemiluminescence detection, image acquisition and analysis were performed using Applied Biosystems Chemiluminescence Detection Kit (P/N 4342142) and Applied Biosystems 1700 Chemiluminescent Microarray Analyzer (P/N 4338036) following the manufacturer's protocol (P/N 4339629). Images were auto-gridded and the chemiluminescent signals were quantified, corrected for background, and finally, spot and spatially-normalized using the Applied Biosystems 1700 Chemiluminescent Microarray Analyzer software v 1.1 (P/N 4336391). For inter-array normalization, we applied Quantile normalization across all microarrays to achieve the same distribution of signal intensities for each array.

### SAM Analysis

Significance analysis of microarrays (SAM; available at the world wide web stat.stanford.edu/tibs/SAM/, and see Tusher et al., Proc Natl Acad Sci USA 98, 5116-21 (2001)) was used to determine potential signature genes distinguishing TAA from control samples, or distinguishing ascending TAA from descending TAA samples.

### Hierarchical Clustering Analysis

Average-linkage hierarchical clustering analysis using centered correlation analysis and visualization was performed using the CLUSTER and TREEVIEW programs (software available at the world wide web genomewww5.stanford.edu/resources/restech.shtml).

### PANTHER™ Protein Classification System Analysis

Similar to Gene Ontology™ (GO), PANTHER™ (Protein ANalysis THrough Evolutionary Relationships) Protein Classification System (Applied Biosystems, Foster City, Calif. world wide web panther.appliedbiosystems.com) classifies proteins in families/sub-families, molecular functions, biological processes and biological pathways. Molecular functions, biological processes and biological pathways over-represented by expression profile genes of the TAA were identified and the statistical significance of the overrepresentation was quantified by a random overlapping p value using the binomial test with all the genes represented by the Applied Biosystems Human Genome Survey Microarray as the reference list (Cho et al., Trends Genet 16, 409-15 (2000)). Bonferroni correction for multiple testing was also used for determining significance in molecular function and biological process.

### Construction and Validation of Prediction Models for Risk Assessment of TAA

A 61-sample training set containing 36 TAA patients (24 males and 12 females) and 25 controls (7 males, 18 females) were used to select classifier genes and construct prediction model. Genes were first filtered based on the criteria that their expression levels are above the detection threshold (Signal to Noise >3) in 50% of samples in either TAA or control group. The resulting 16,656 genes from the filtering were then subjected to further gene selection. The prediction power for each gene was evaluated using bootstrap re-sampling method coupled with two-tailed t-statistics. Specifically, during each bootstrap re-sampling process, equal numbers (n=25) of TAA and control samples were partitioned (repetition allowed) to form a new data set. A two-tailed t statistics was applied to the new data set and the top 500 genes with the most significant p-value were selected. This bootstrap re-sampling process was repeated for 500 times and a total of 500 500-gene lists were generated. Genes were then ranked based on their frequency in appearing in the 500 500-gene lists and genes with frequency >50% and with average ranking >500 were chosen for further analysis (in general about 105-120 genes). Class prediction was performed by using prediction analysis of microarrays (PAM), a statistical package (available on the world wide web www-stat.stanford.edu/˜tibs/PAM/) that applies nearest shrunken centroid analysis for sample classification. The optimal number of classifier genes was determined using 10-fold cross validation method on the training set. The 61 (training) samples are partitioned into 10 bins, with equal representation of TAA and controls as the initial set of samples. Nine bins are used for learning purposes to generate an ordered gene list (as described herein) based on the gene probability to be ranked in top 500 most discriminative genes. For any set of top 1, 2, 3, . . . n genes of this ordered list of genes, prediction models are built using the 9 (learning) bins and TAA status of samples belonging to the remaining bin is predicted. Using clinical diagnostics as the reference, True Positives (TP), True Negative (TN), False Positive (FP), and False Negative (FN) were calculated. The prediction performance was evaluated using the following statistics:

Sensitivity=TP/(TP+FN)

Specificity=TN/(FP+TN)

Accuracy=(TP+TN)/(TP+FP+TN−FN)

### TaqMan® Assay-Based Real Time PCR Validation

Out of 94 samples analyzed by microarrays, only 82 samples (52 training samples, 30 testing samples) have enough RNA samples to perform TaqMan® real-time PCR validation. mRNA expression of 71 genes was measured in each of the 82 samples by real-time PCR using TaqMan® Gene Expression Assays and the ABI PRISM 7900 HT Sequence Detection System (Applied Biosystems, Foster City, Calif.). 30 ng of total RNA from each sample was used to generate cDNA using the ABI High Capacity cDNA Archiving Kit (Applied Biosystems, Foster City, Calif.). The resulted cDNA was subjected to a 14-cycle PCR amplification followed by real-time PCR reaction using the manufacturer's TaqMan® PreAmp Master Mix Kit Protocol (Applied Biosystems, PN 4366127). Four replicates were run for each gene for each sample in a 384-well format plate. Among the two measured endogenous control genes (PPIA (Alias: cyclophilin A) and MGB2) we chose PPIA for normalization across different genes based on the fact that this gene showed the most relatively constant expression in different breast carcinomas (data not shown). Based on TaqMan data, the coefficient of the 41 classifier genes were re-learned from the 52 training samples and used to predict the 30 testing samples using the same method applied to microarray data.

### GEO Accession

The complete data sets of this study including both microarray and TaqMan data can be accessed from GEO (world wide web ncbi.nlm.nih.gov/projects/geo/)

The present teachings provide novel methods, compositions, and kits for detecting and treating aortic aneurysms. Thoracic aortic aneurysm (TAA) is usually asymptomatic and is associated with high mortality. Adverse clinical outcome of TAA is preventable by elective surgical repair. However, identifying at-risk individuals is difficult. We hypothesized that gene expression patterns in peripheral blood cells may correlate with TAA disease status. In the present teachings, we carried out a comprehensive gene expression survey on peripheral blood cells obtained from TAA patients and normal individuals. We generated a distinct molecular signature in peripheral blood cells that can classify TAA patients from normal individuals. Validated by TaqMan® based real-time PCR, the classifier genes provided by the preset teachings define a set of promising potential diagnostic markers, providing for a blood-based gene expression test to facilitate early detection of TAA disease. Furthermore, the biological pathways associated with the signature genes of TAA provide further insights into the molecular pathogenic process of this disease.

### Gene Expression Signature of Thoracic Aortic Aneurysm in Peripheral Blood

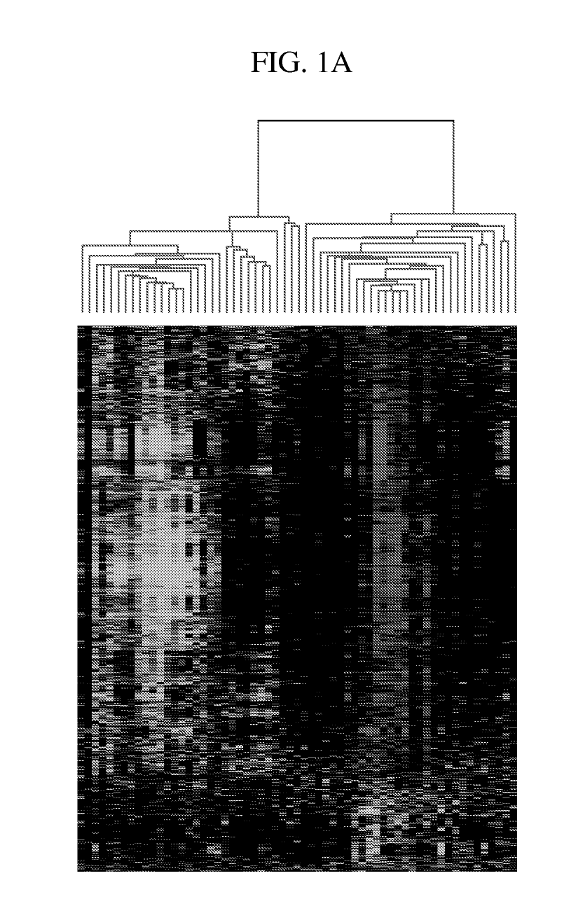

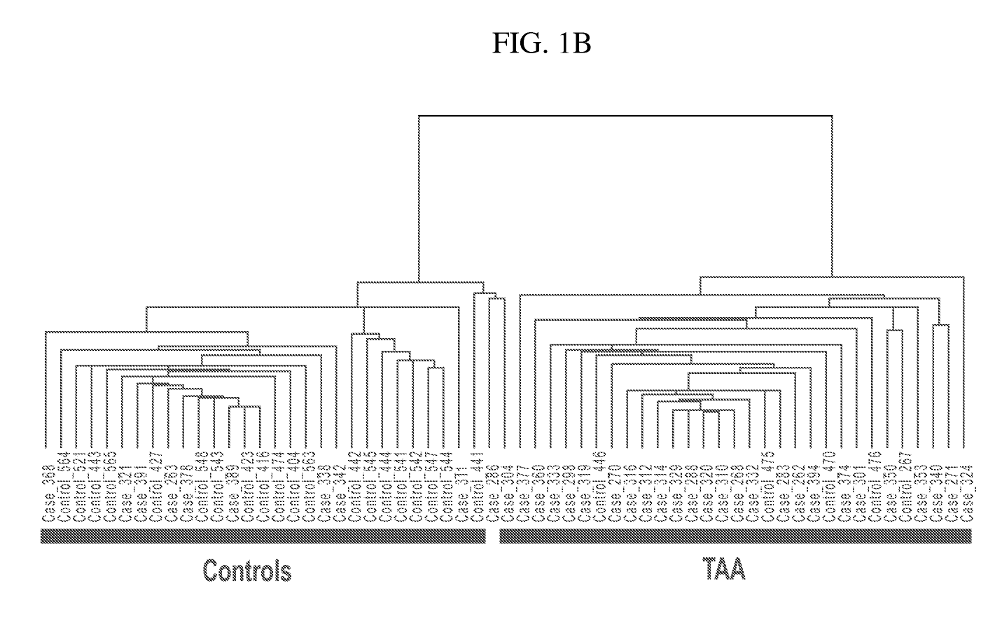

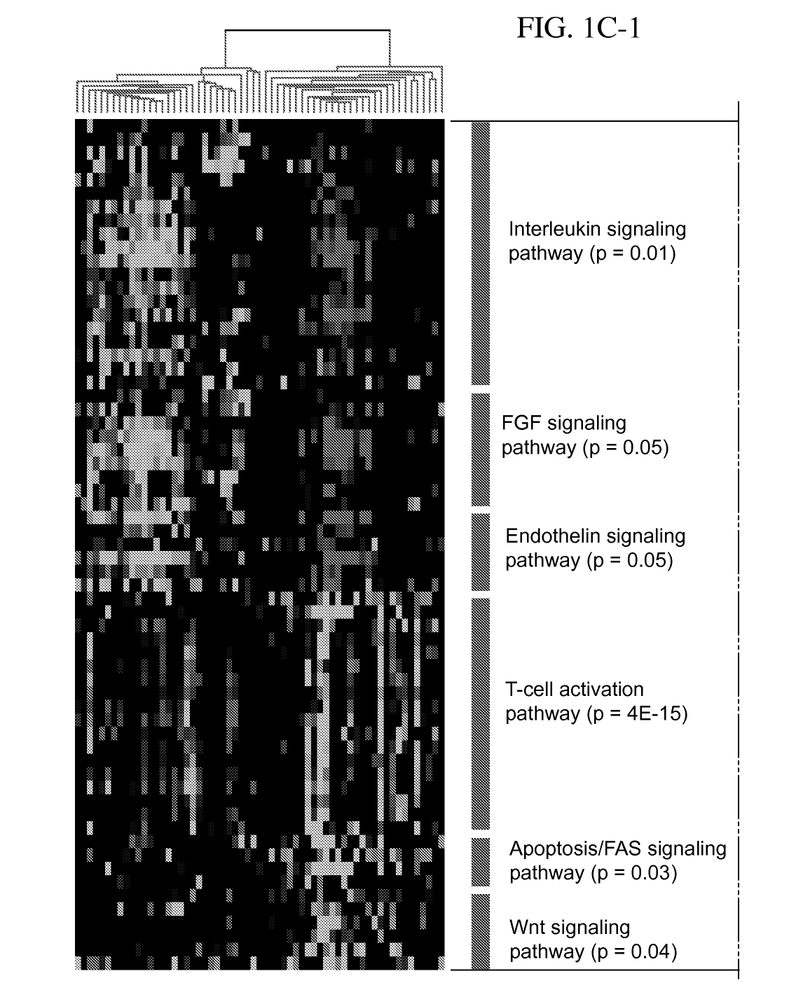

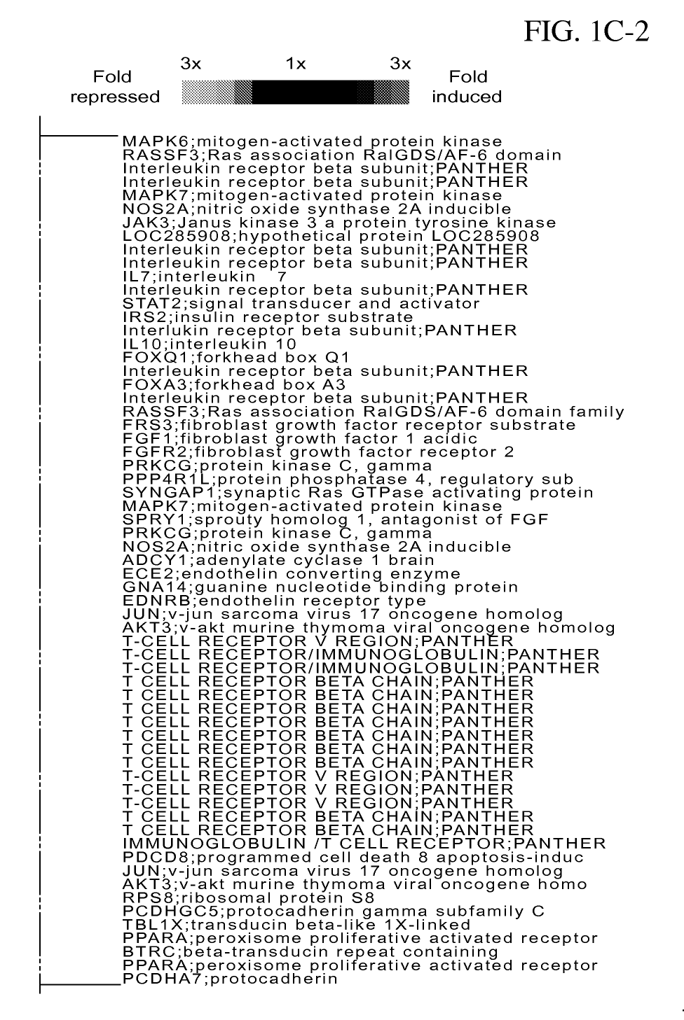

Peripheral blood cells from a total of 58 TAA patients and 36 spousal controls were analyzed in this study. The complete clinical profile of patients and controls, as well as their smoking history, were recorded. Complete blood cell counts, including WBC, neutrophils, lymphocytes, monocytes, eosinophils and basophils, were determined in all blood samples collected from TAA patients and controls. None of these specific cell counts demonstrated significant association with the TAA disease status based on logistic regression analysis. To explore whether we can identify a gene expression signature of TAA disease from peripheral blood samples, gene expression profiles of 61 whole-blood RNA samples (training set) collected from 36 TAA patients and 25 controls were analyzed using the Applied Biosystems Human Genome Survey Microarrays representing 29,098 individual human genes. Using SAM (Significance Analysis of Microarray) analysis, 1207 genes were identified as significantly differentially expressed genes between the TAA and control groups based on the following criteria: (1) False Discovery Rate (FDR)<4% from 300 permutation testing; and (2) average fold change between the TAA patients and controls more than 1.3-fold. To examine whether the imbalanced gender distribution within the TAA and control groups may confound the identification of TAA signature genes, SAM analysis was performed between the 31 male and 30 female samples within the training set. 28 genes were identified as gender-specific genes using the same criteria (FDR<4% and >1.3-fold between the two gender groups). Not surprisingly, 21 out of the 28 gender-specific genes were found located on either Y or X chromosome. 8 of the 28 gender specific genes (5 Y-linked and 3-X linked) were part of the 1207 differentially expressed gene list and were excluded in further analysis. FIG. 1A displays hierarchical clustering diagrams of the 61 whole-blood RNA samples using the remaining 1199 potential TAA signature genes, among which, 988 genes were specifically over-expressed and 211 genes were specifically under-expressed in TAA samples when compared to the control samples (FIG. 1A, the complete gene list is also available. These signature genes generally clustered the TAA patients and the controls into two distinct branches with only a few exceptions (FIG. 1B).

To explore potential molecular mechanisms underlying these TAA signature genes, we analyzed which biological pathway was significantly over-represented by these signature genes using the PANTHER™ Protein Classification System analysis (Mi et al., Nucleic Acids Res 33, D284-8 (2005)). As shown in FIG. 1C, among the 988 genes that are up-regulated in the TAA patients, the most over-represented biological pathways are associated with interleukin signaling activities (p-value=0.01), FGF signaling pathway (p-value=0.05), and Endothelin signaling pathway (p-value=0.05). The over-expressed genes within the interleukin signaling pathway represent various components of cellular immune responses, including cytokines IL7 and IL 10, cytokine receptors IL11AR, forkhead transcriptional factors (FOXQ1, FOXA3) and downstream signal transduction genes, MAPK6, MAPK7 and STAT2. In a similar fashion, we also analyzed which cellular pathways may underlie the 211 down-regulated genes in TAA patients. As shown in FIG. 1C, the most overrepresented pathways associated with the down-regulated genes in TAA samples include T-cell activation (p-value=4E-15), FAS-mediated apoptosis (p-value=0.03) and Wnt signaling pathways (p-value=0.04).

### Molecular Profiles Characterizing Subtypes of TAA

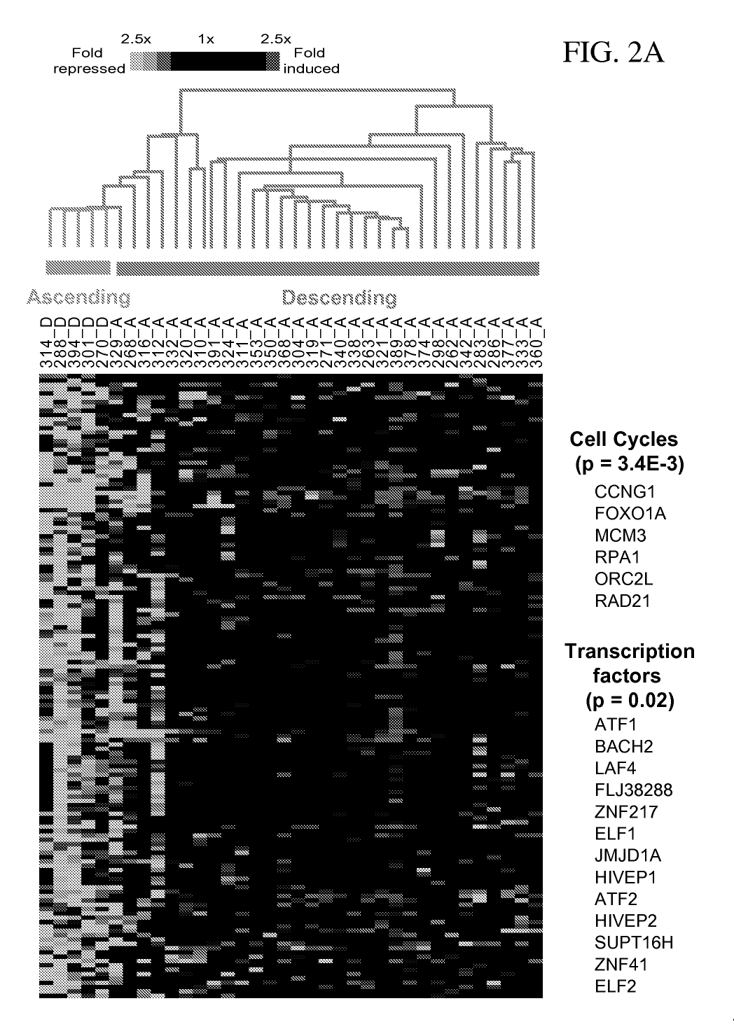

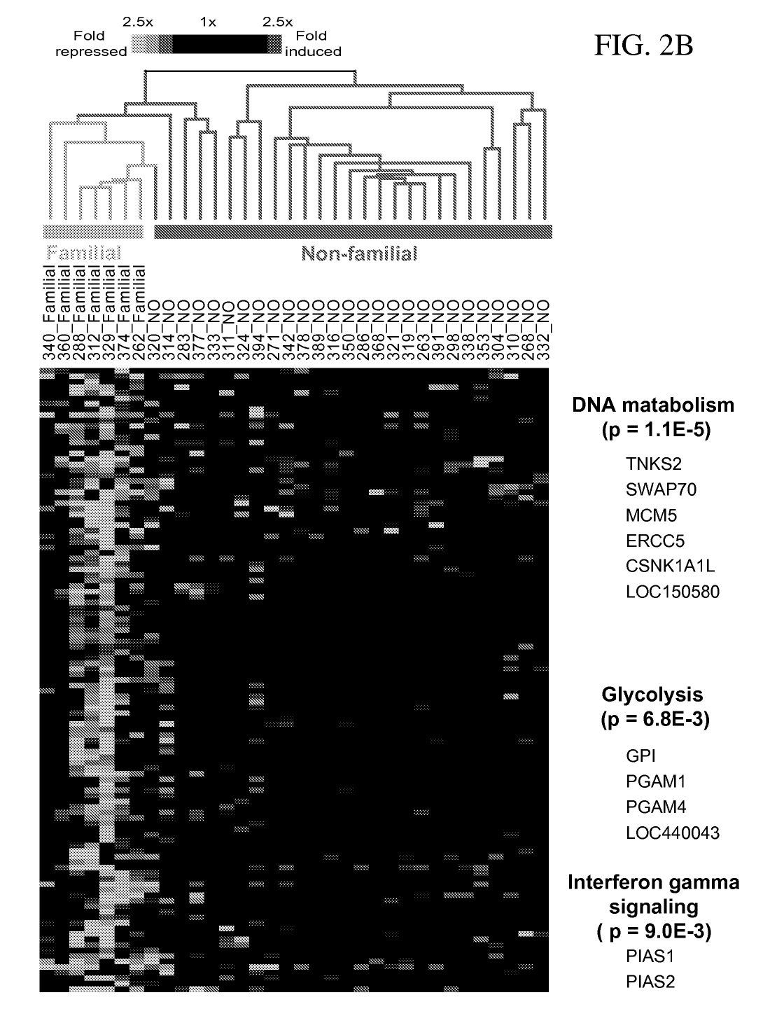

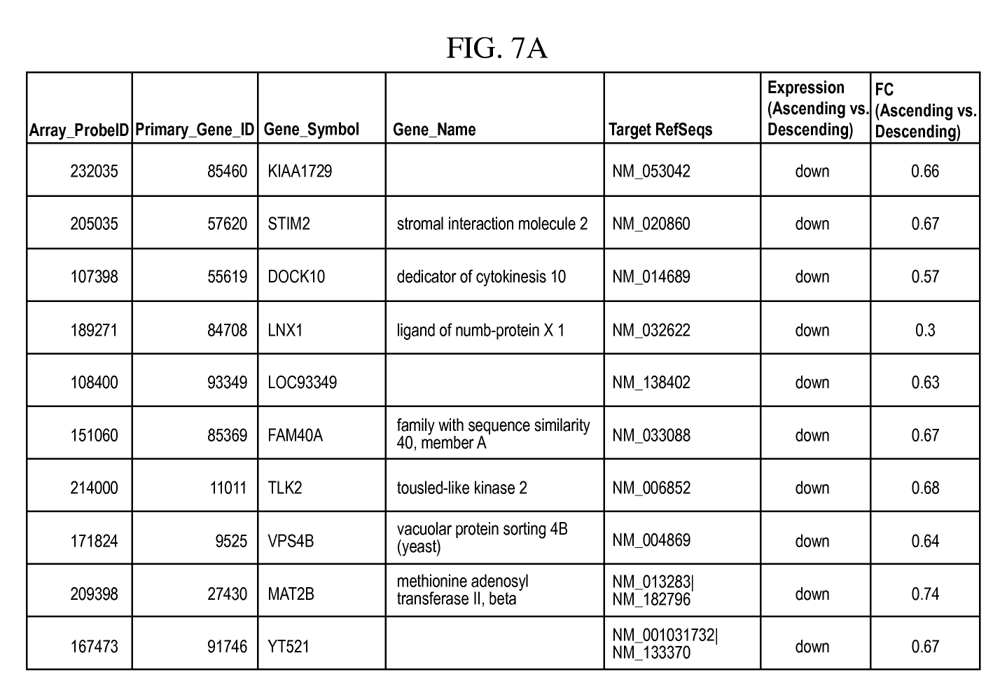

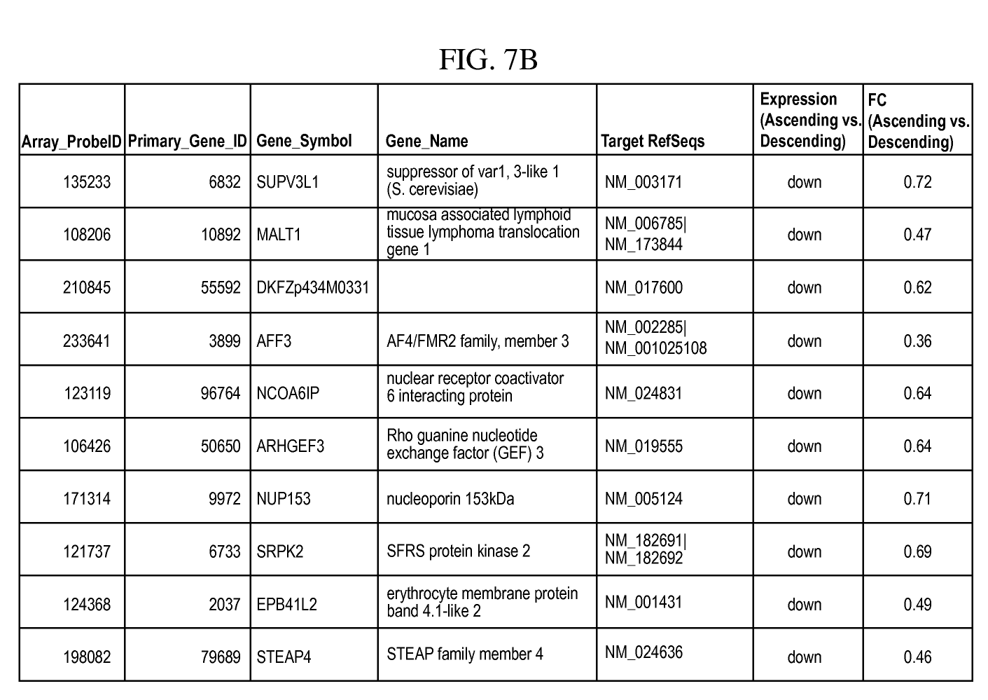

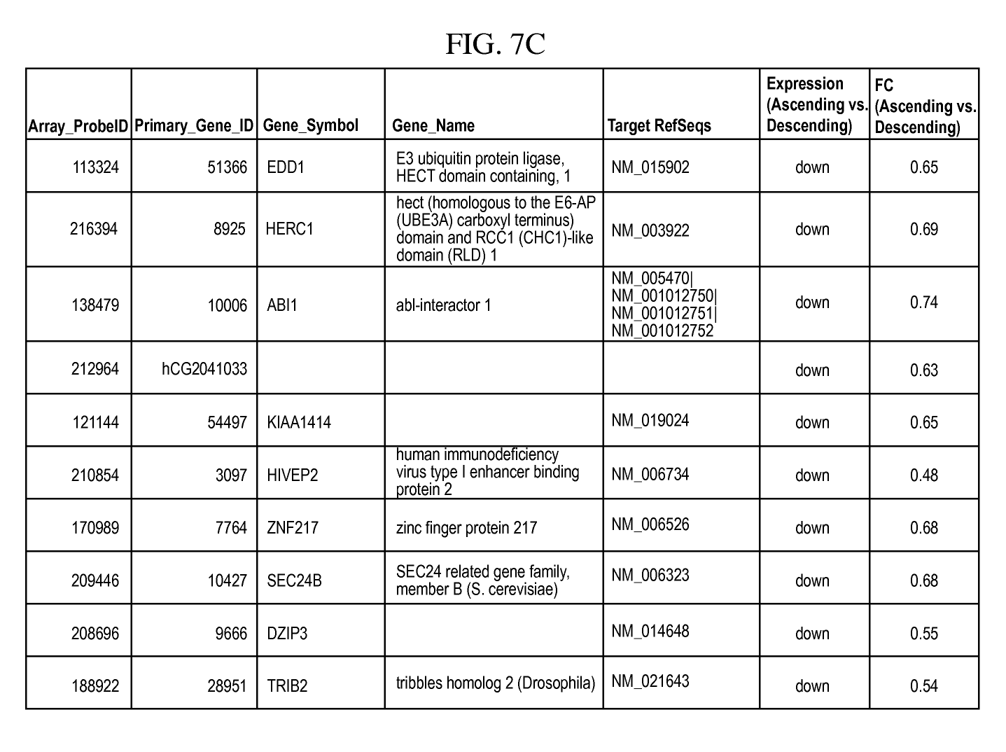

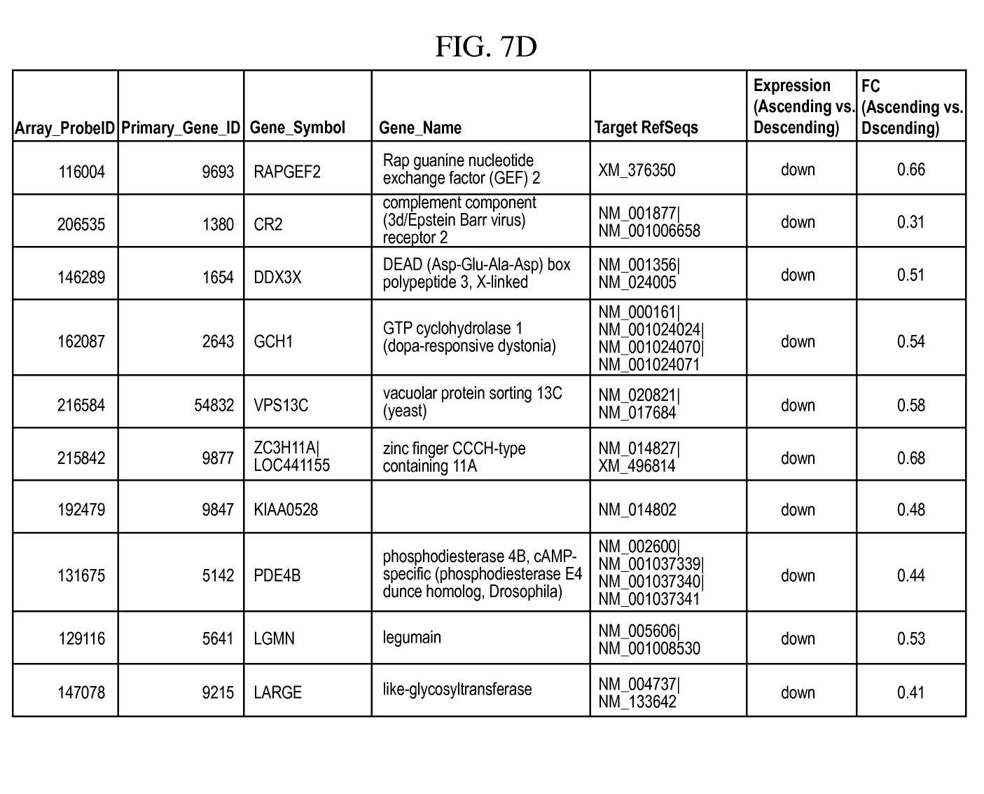

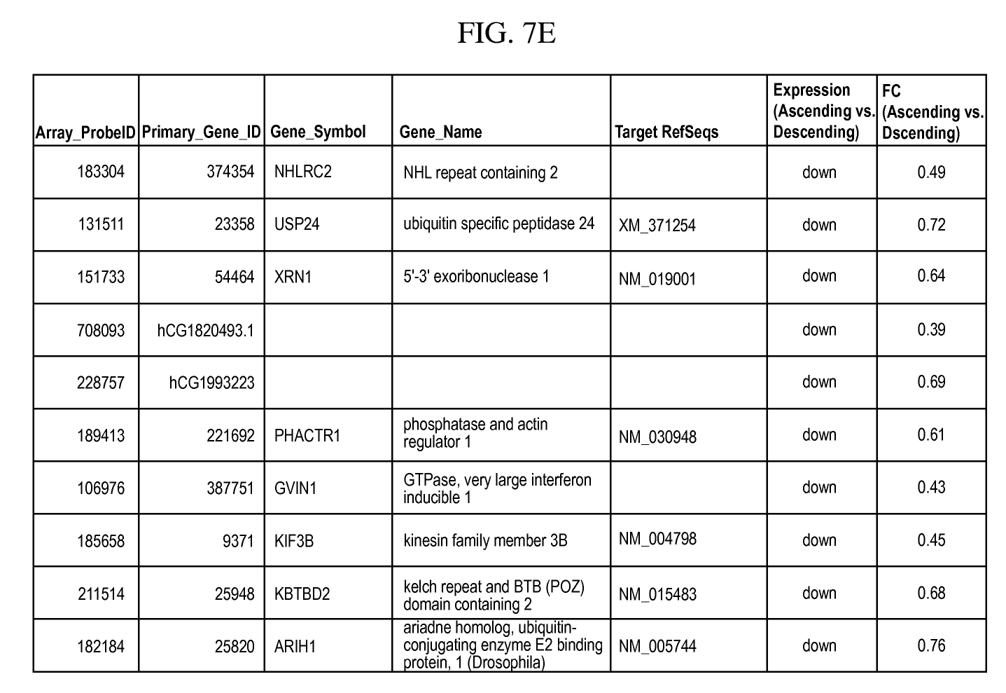

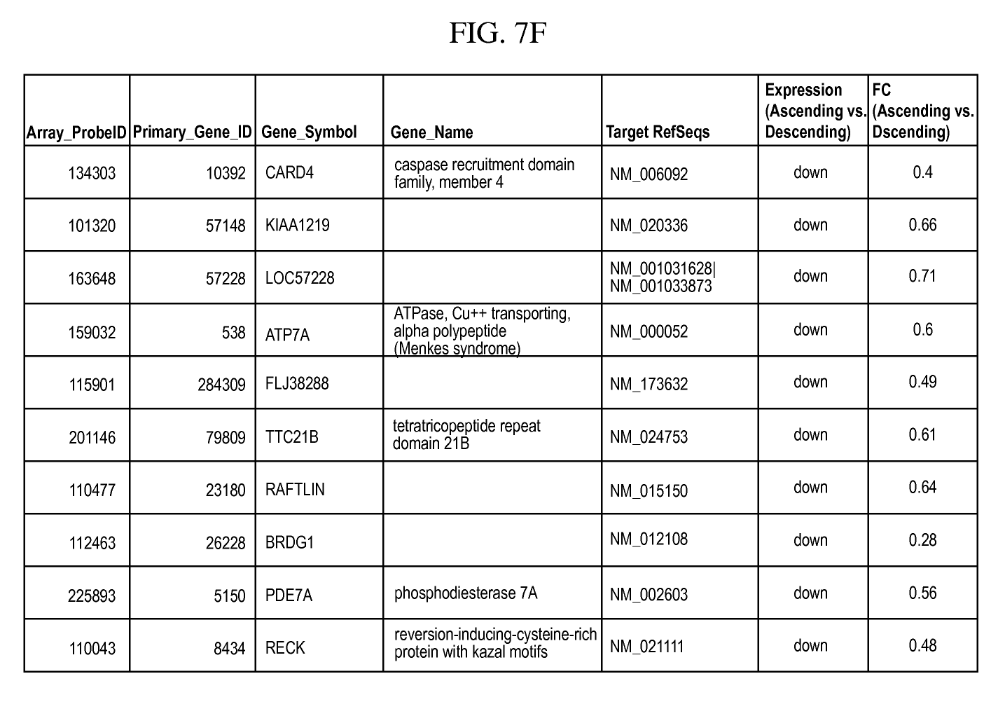

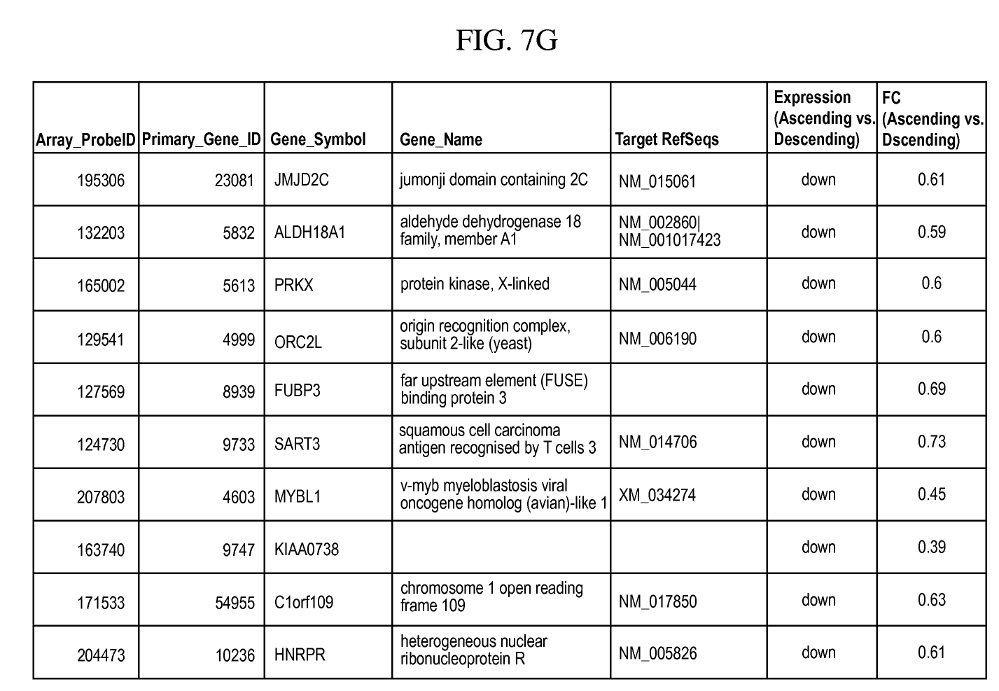

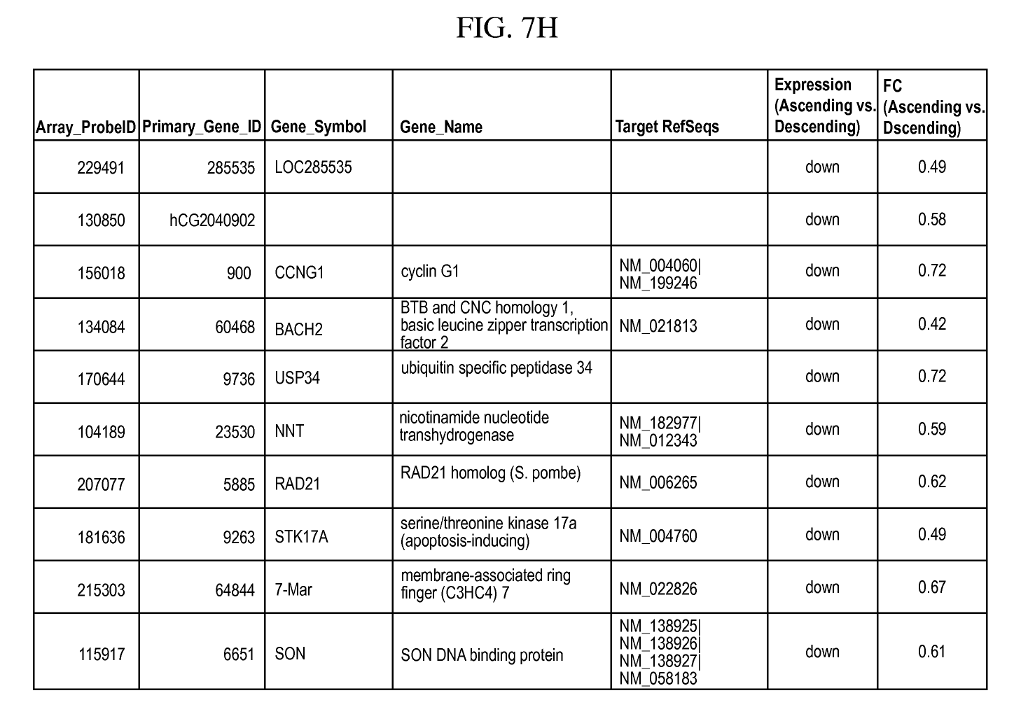

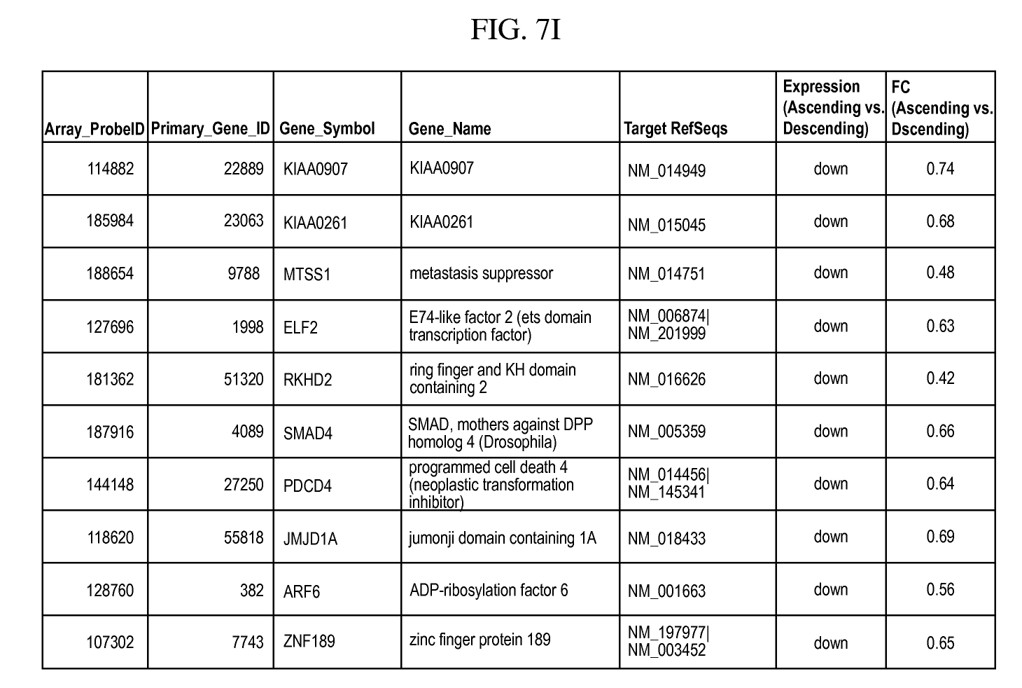

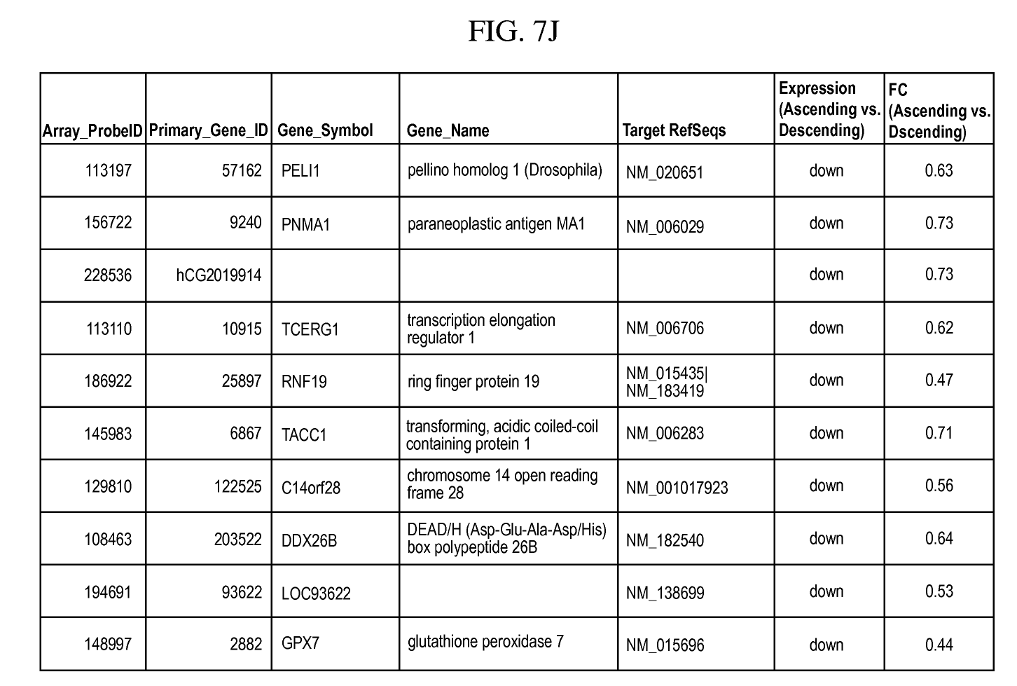

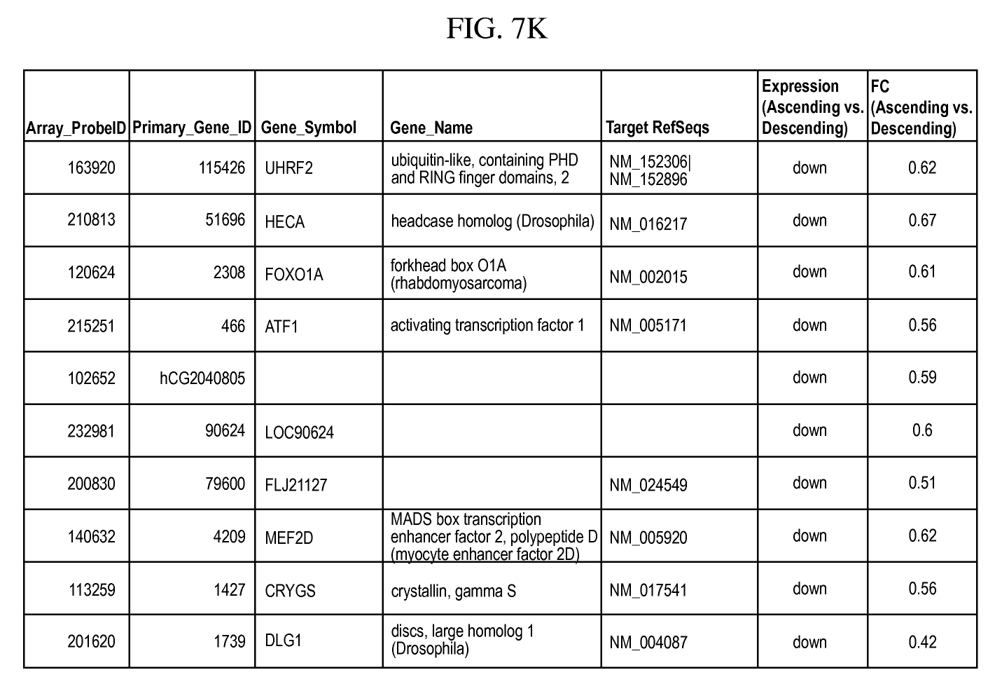

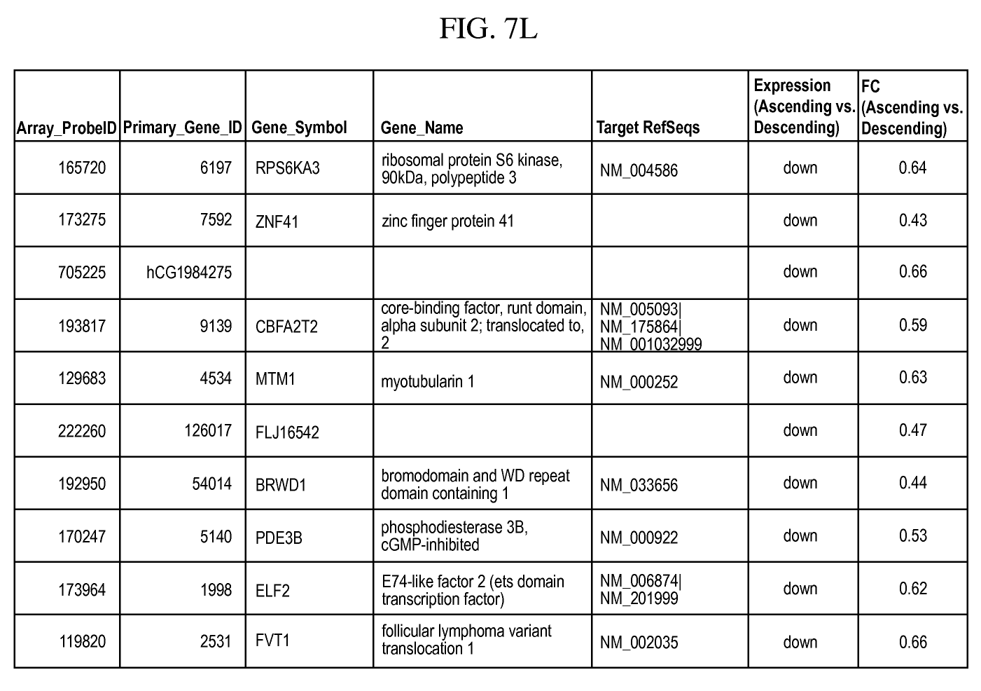

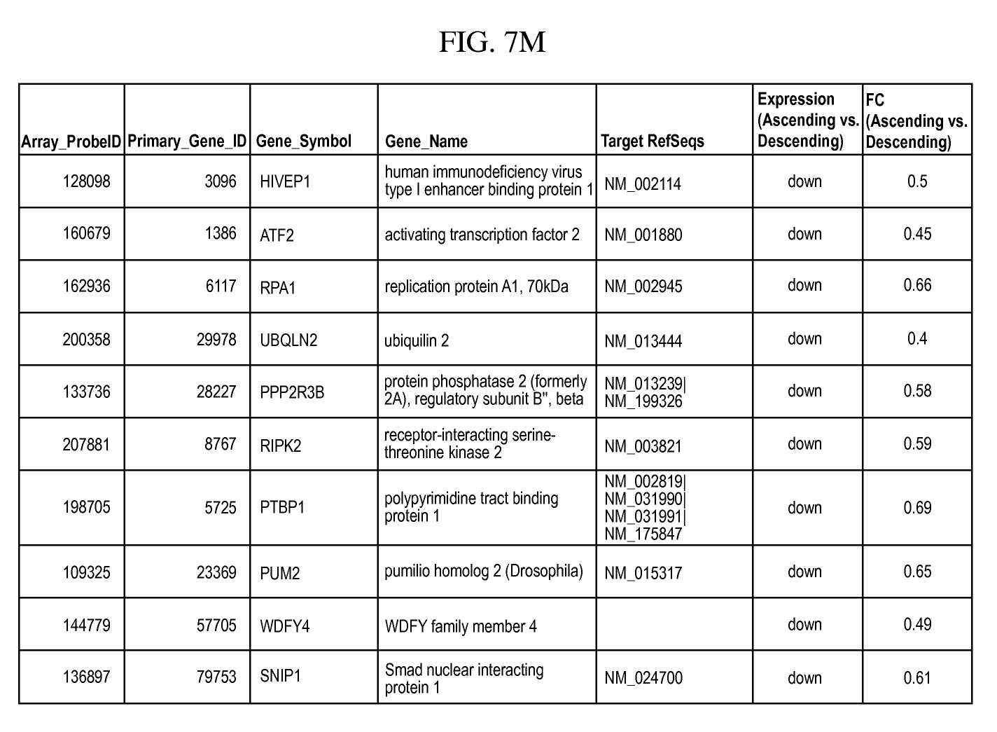

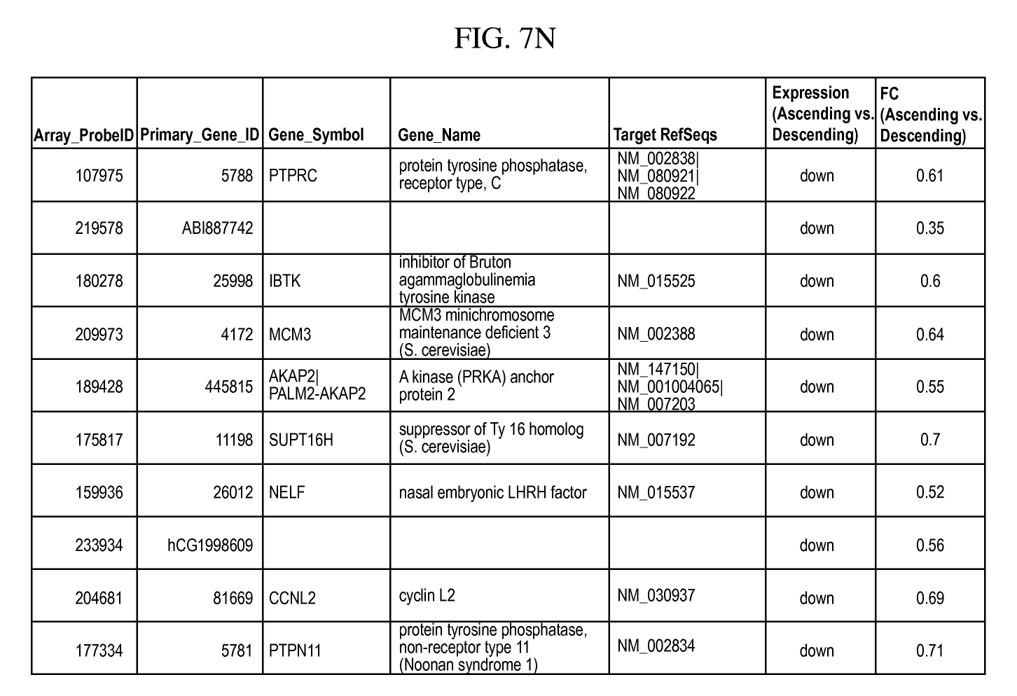

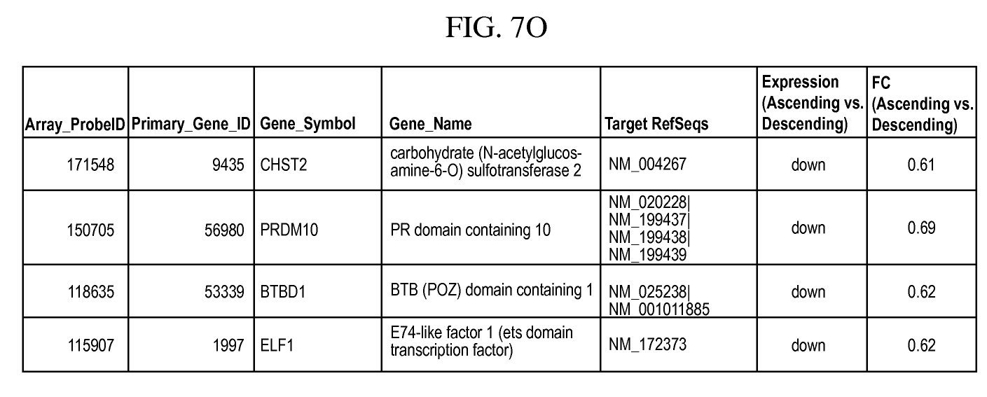

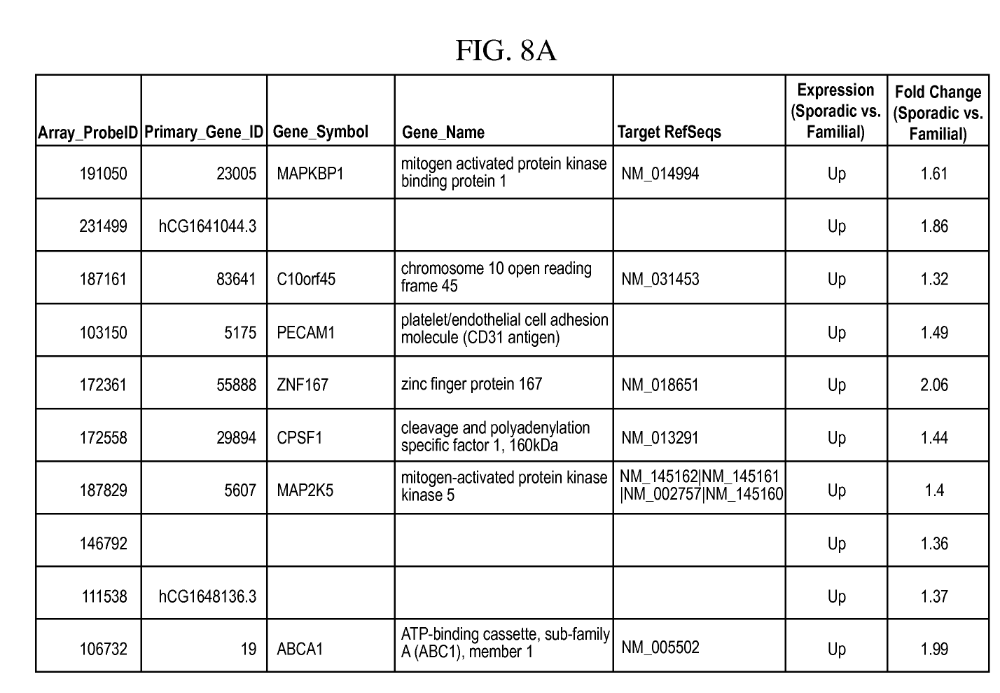

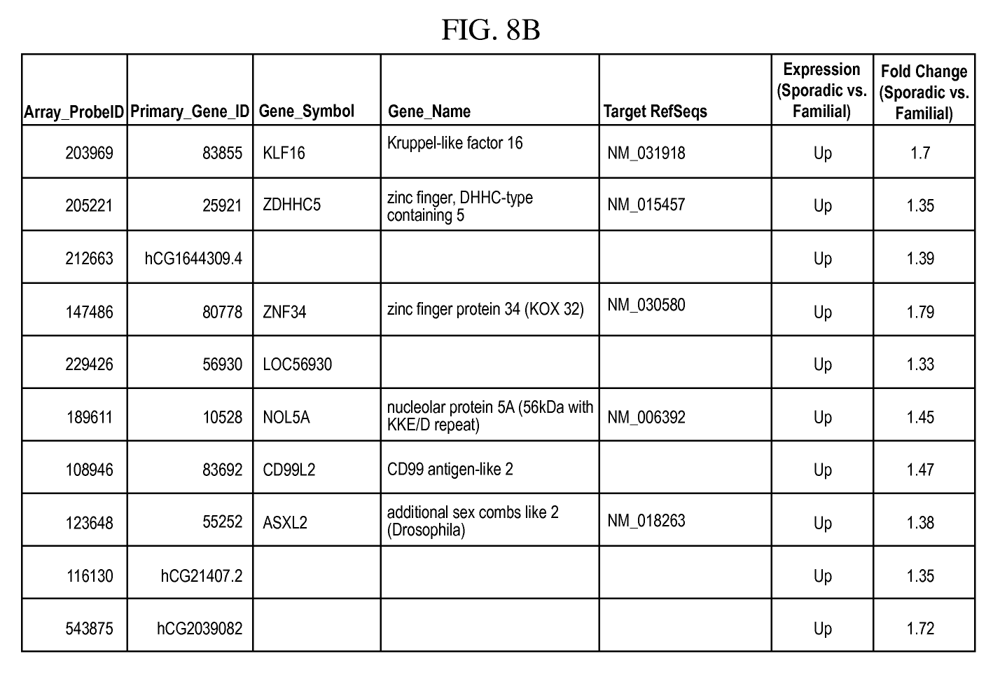

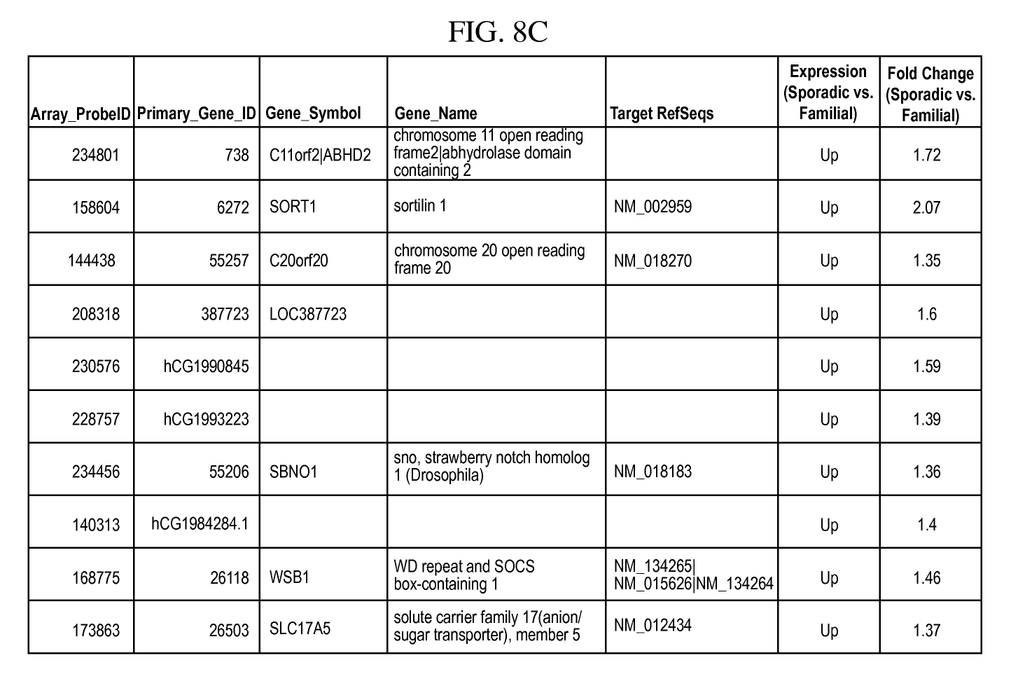

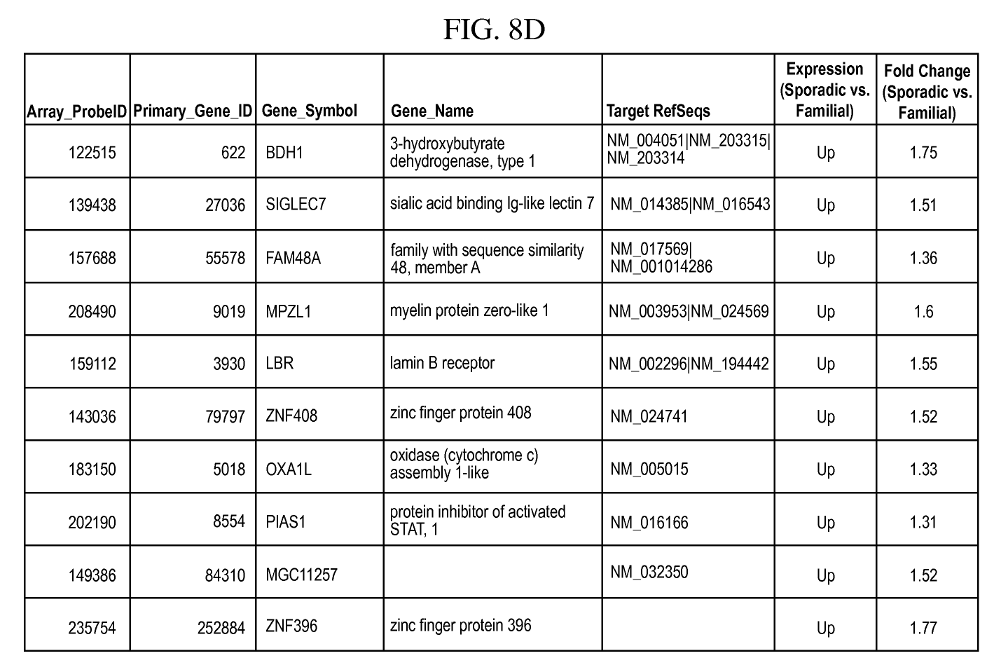

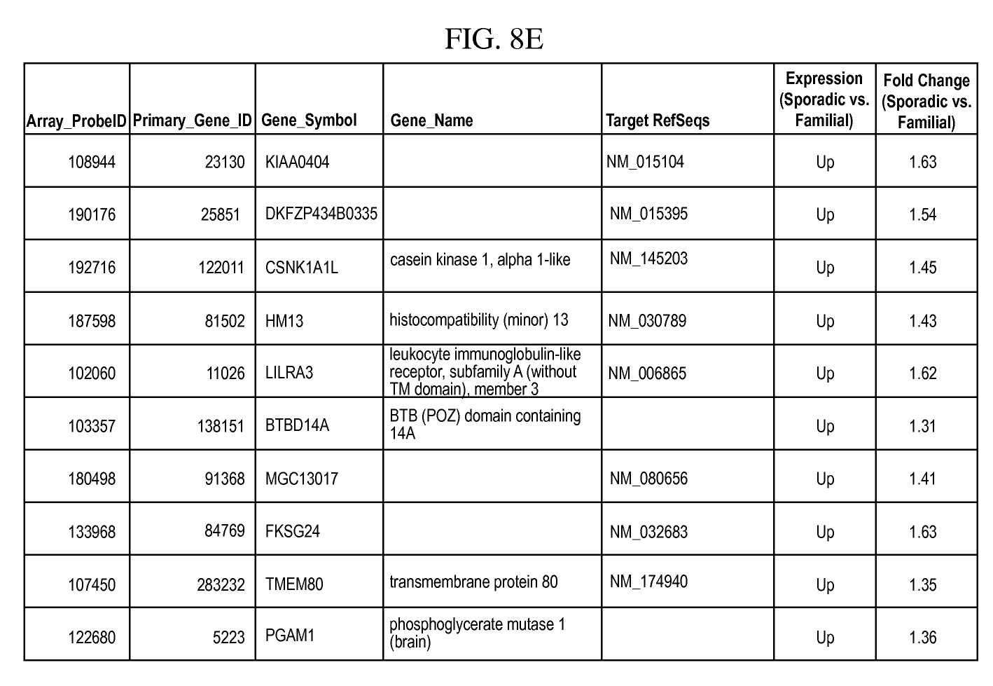

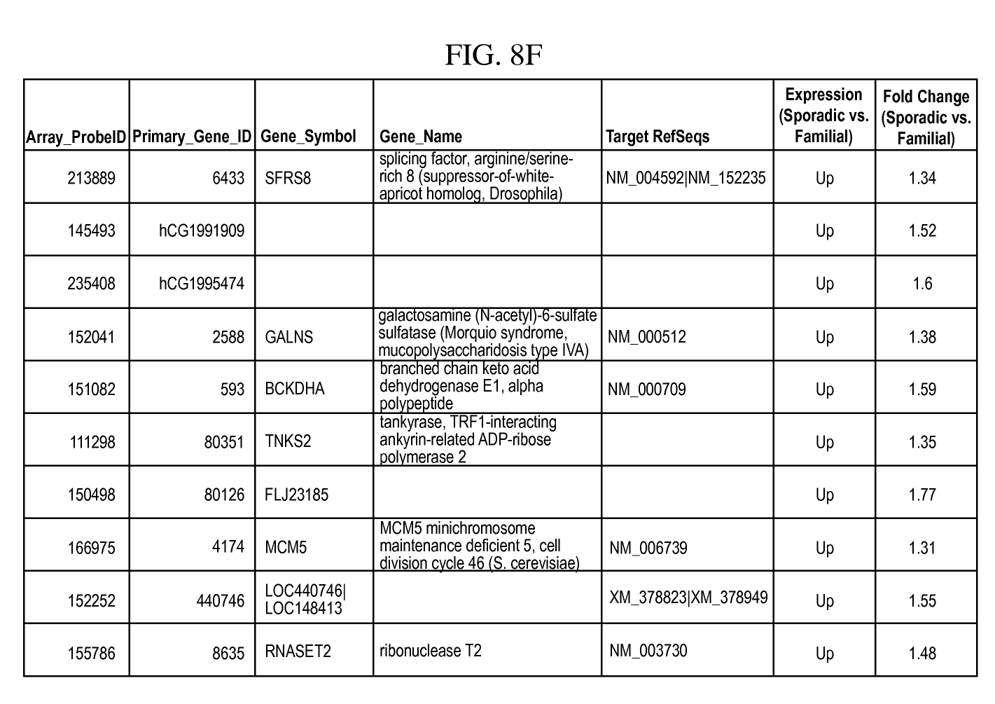

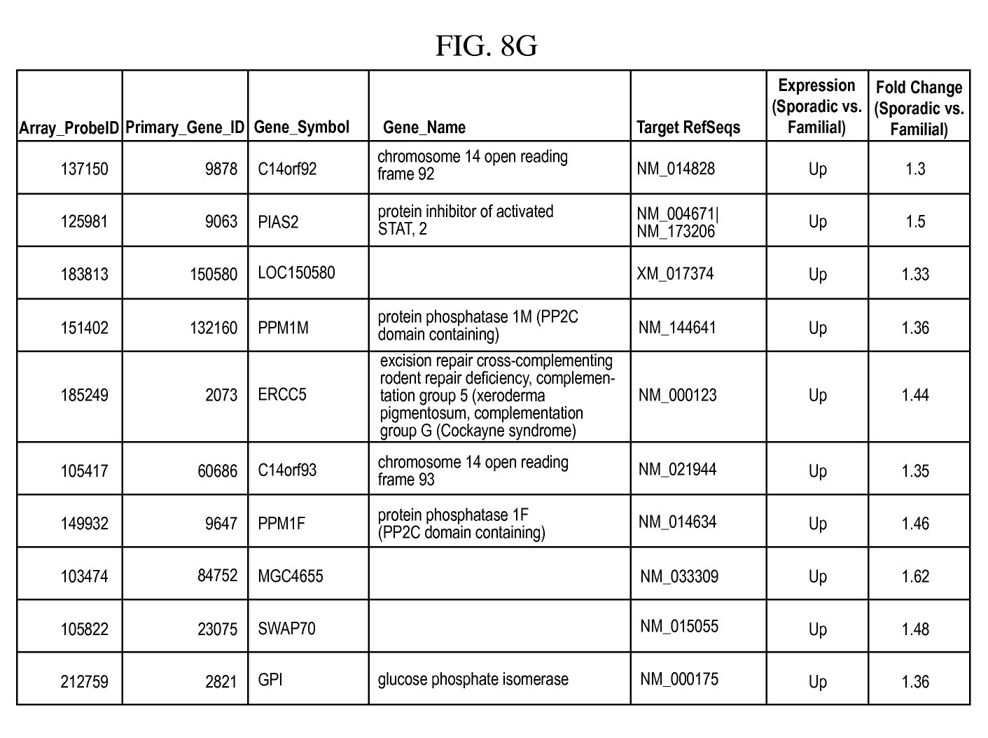

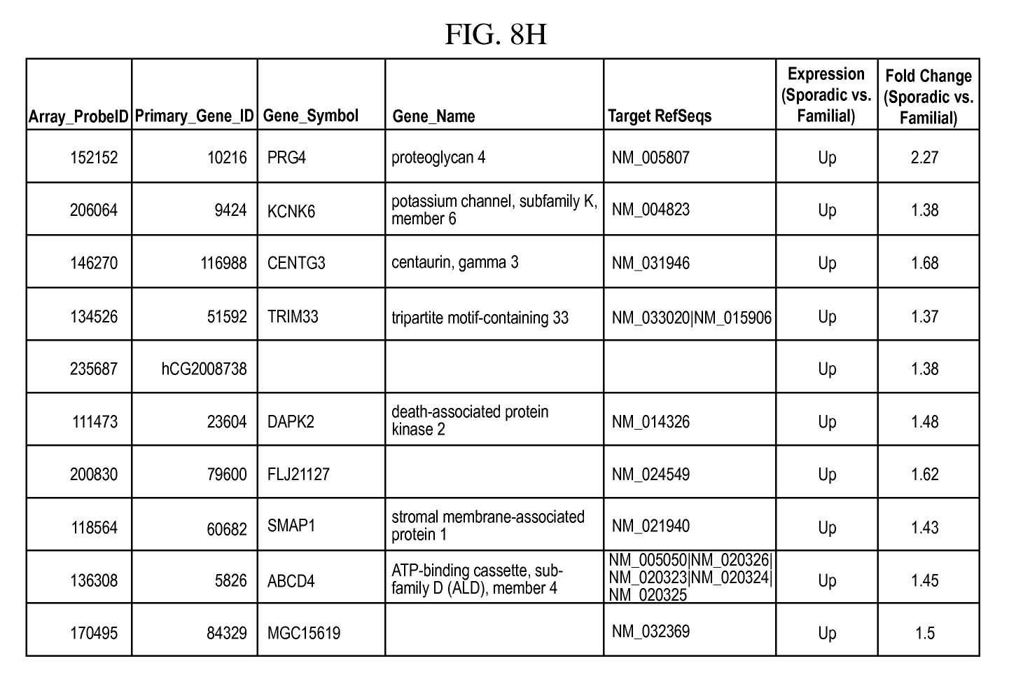

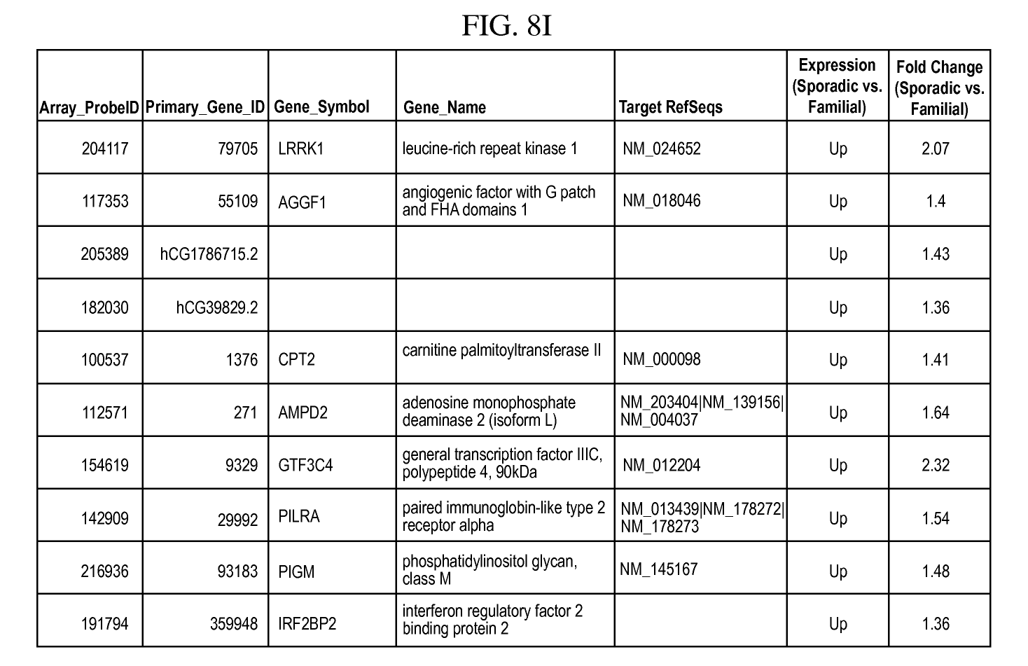

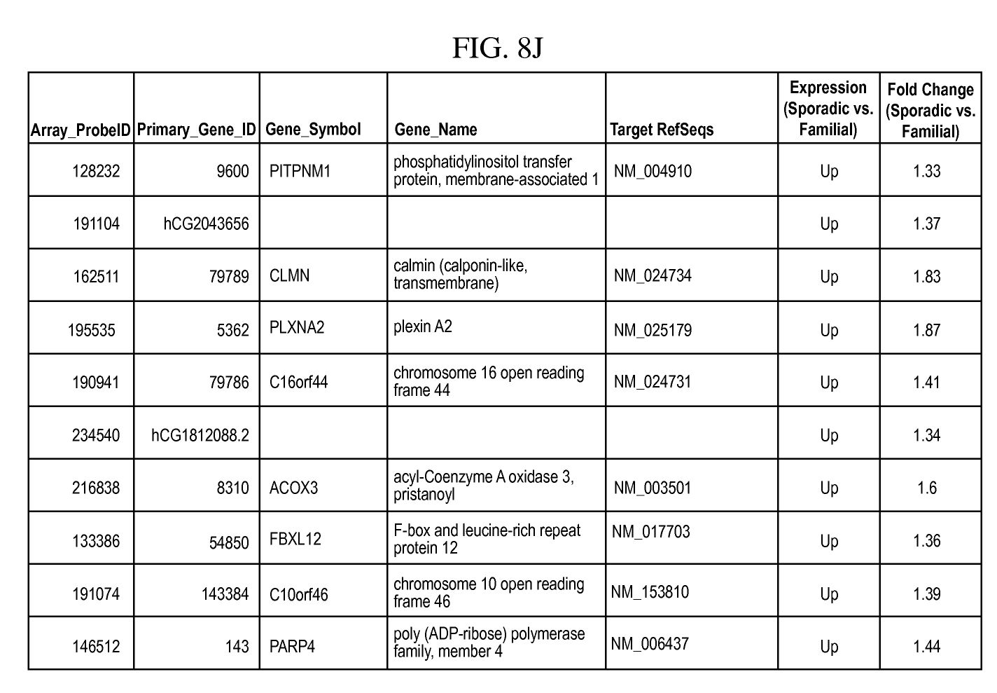

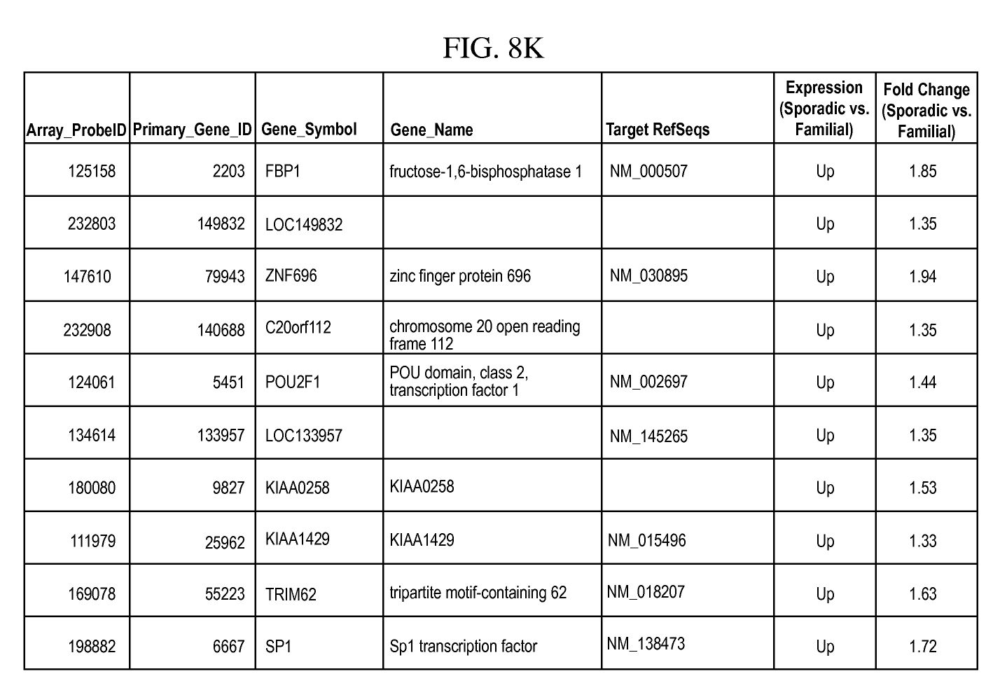

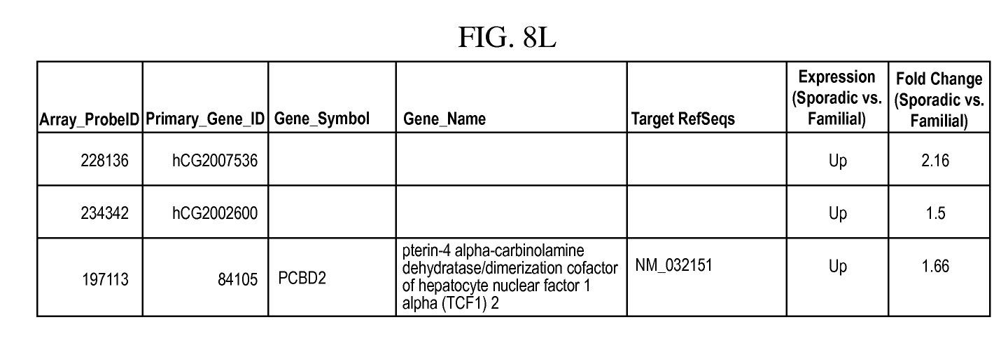

One of the main clinical characteristics of TAA is the location of the aneurysm-in the ascending or descending aorta. Aneurysms in these two locations are felt to represent very different clinical phenomena (Albornoz et al., Ann Thorac Surg 82, 1400-5 (2006)). The location of an aneurysm is distinctly connected with the embryology, pathogenesis, clinical course, and treatment patterns of a thoracic aneurysm. To explore whether we can identify signature expression profiles characterizing location-specific TAA, SAM analysis was performed on 36 TAA samples within the training set (31 ascending vs. 5 descending). 144 genes were significantly over-expressed in the ascending TAA samples (FDR<2%, FC>1.3) (see FIG. 7). Hierarchical clustering analysis of these 144 genes clustered the descending TAA separately from the ascending TAA samples (FIG. 2A). PANTHER™ Protein Classification System analysis revealed that genes involved in cell cycles (e.g. RAD21, ORC2, LRPA1, MCM3, FOXO1A, and CCNG1) are significantly over-represented in the 144 ascending TAA differentially expressed genes (p-value=3.4E-3). Thirteen transcription factors were also found significantly over-expressed in the ascending TAA samples (p value=0.02, FIG. 2A), including ELF1 and ELF2, two transcriptional factors involved in the platelet derived growth factor (PDGF) signaling pathway. PDGF signaling plays a critical role in cellular proliferation and development. Familial aggregation studies indicate that up to 20% of patients with TAA who do not have Marian syndrome (MFS) have a first-degree relative with the disease. To investigate potential gene expression signatures characterizing familial and sporadic TAA, we performed SAM analysis on 7 TAA with family history and 27 TAA patients without family history within the training set. 113 genes were identified as significantly up regulated in sporadic TAA patients (ave FC>1.3 and FDR<4%) and hierarchical clustering based on these genes clustered the familial and sporadic TAA patients into distinct branches (FIG. 2B) (FIG. 8). PANTHER™ Protein Classification System analysis identified the most over-represented biological processes underlying these genes to be involved in various aspects of DNA metabolism (p-value=1.1E-6), including DNA replication (LOC150580, MCM5, TNKS2), DNA repair (ERCC5, CSNK1A1L) and DNA recombination (SWAP70). Several genes involved in glycolysis (PGAM1 and GPI) and interferon signaling (PIAS1 and PIAS2) were also significantly up-regulated within the sporadic TAA peripheral blood. Another interesting gene specifically down-regulated in familial TAA is the angiogenic factor AGGF1. This gene, located at chromosome 5q13.3, is within the previously mapped 5q13-14 locus associated with familial TAA (Guo et al., Circulation 103, 2461-8 (2001); Kakko et al. J Thorac Cardiovasc Surg 126, 106-13 (2003)).

Finally, SAM analysis was also performed to identify signature genes correlated with age, aneurysm size and smoking status of TAA patients, however, no genes were found to correlate significantly with these factors (data not shown).

### Construction of Prediction Model for Risk Assessment of TAA

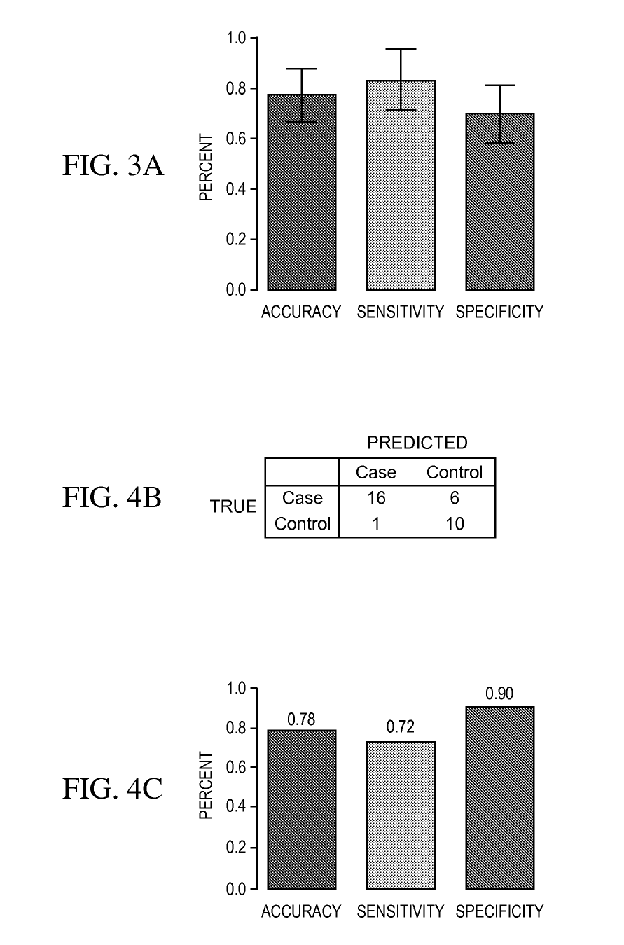

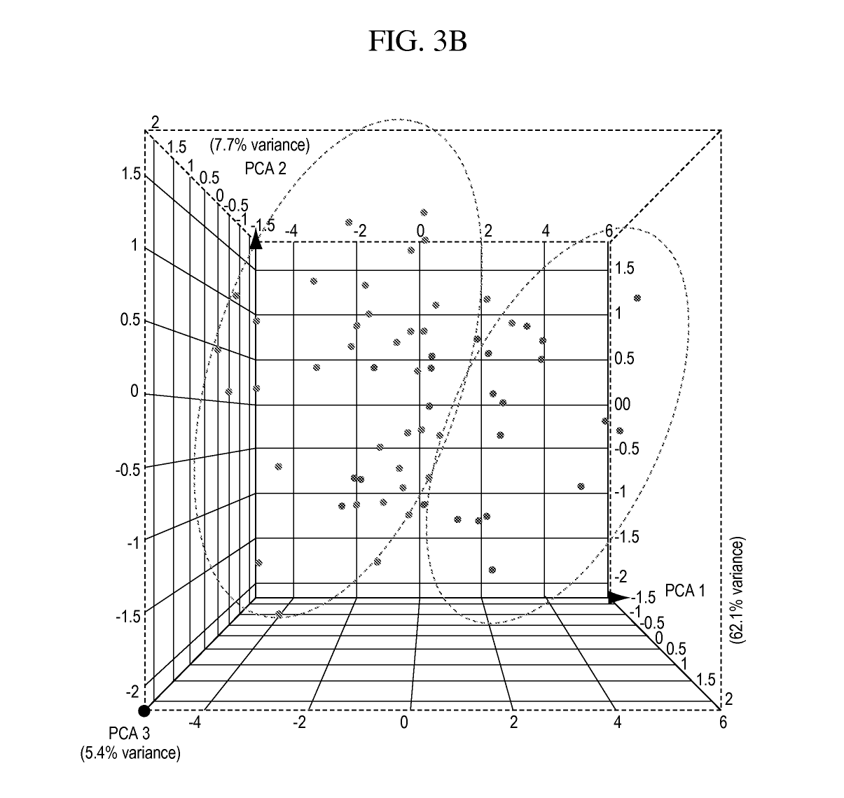

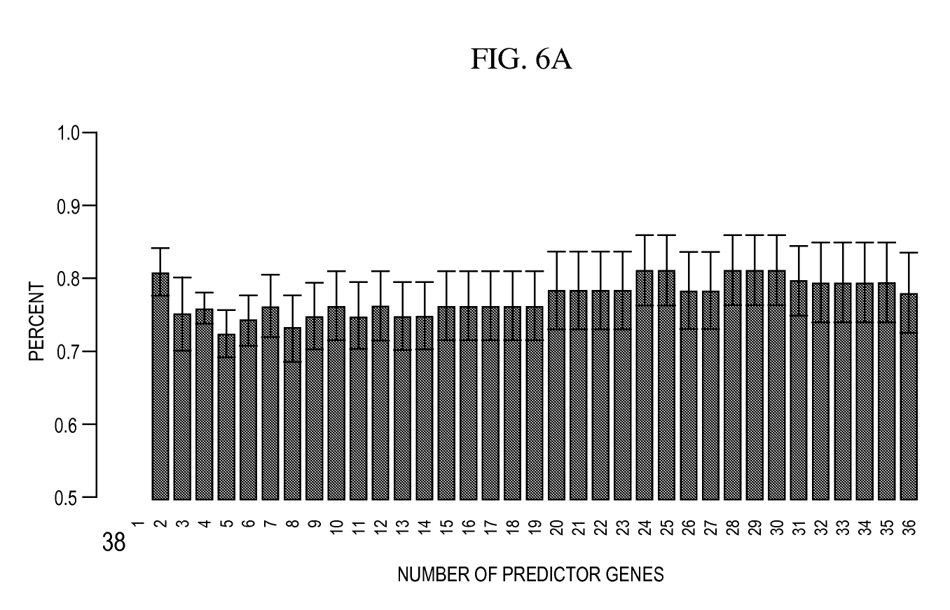

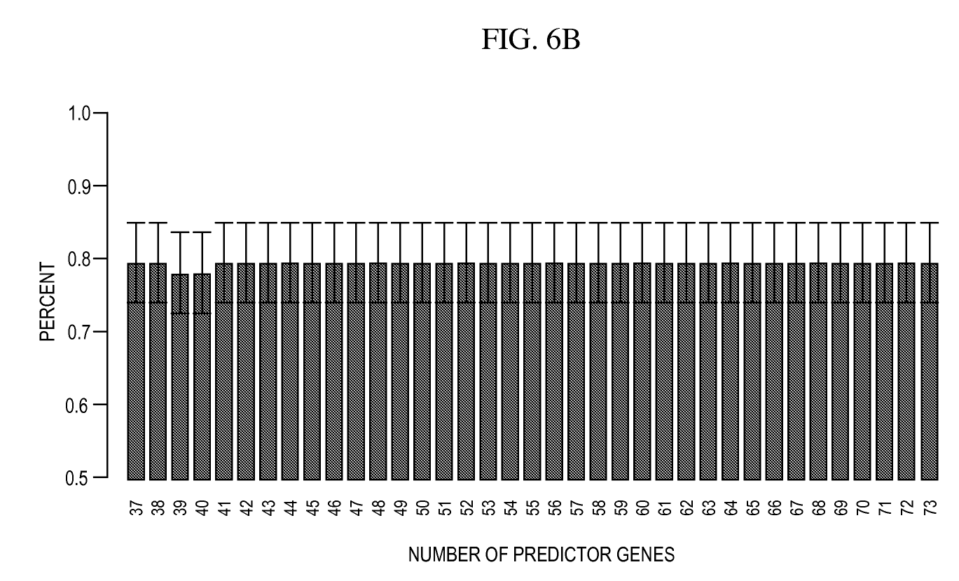

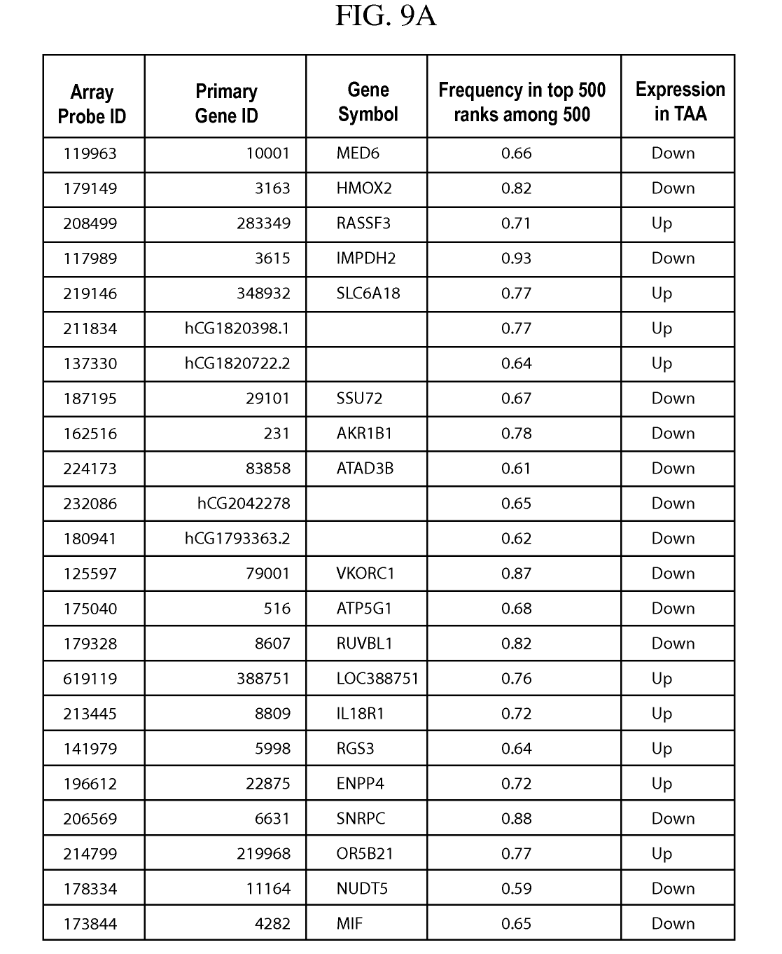

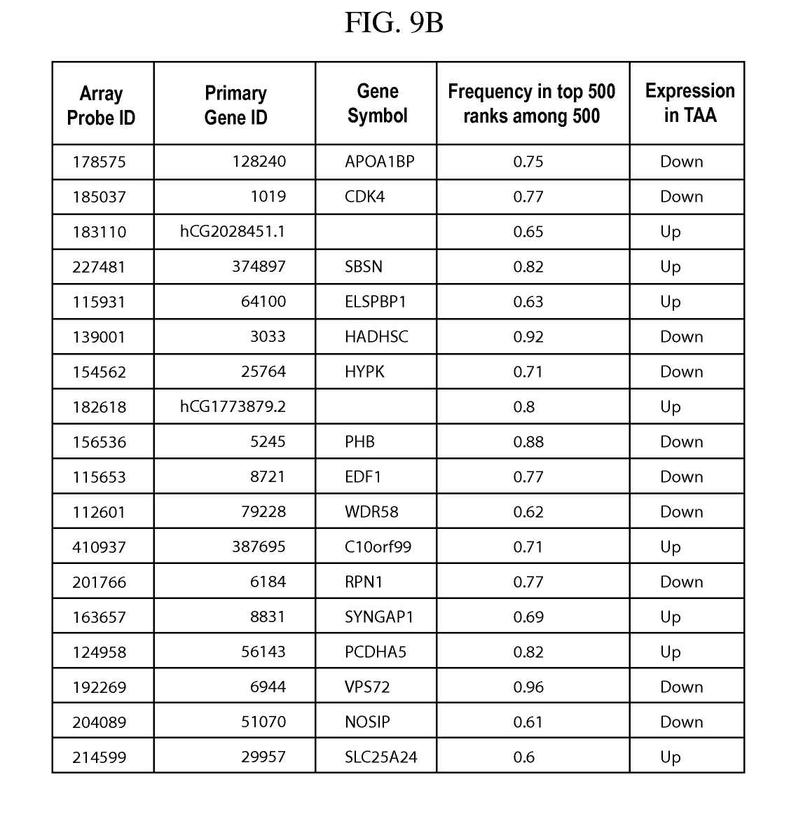

Our goal was to identify a distinct gene expression signature in peripheral blood that may allow development of noninvasive screening tests to identify individuals at risk for TAA disease. Our initial attempt to build a prediction model using PAM analysis yielded poor classification accuracy (data not shown). This may be partially due to the observed higher inter-individual variation associated with the complexity of the TAA disease as well as the heterogeneity of whole blood cells. To overcome this problem, we applied bootstrapping (Efron, B. & Tibshirani, R. J. An Introduction to the Bootstrap. (1993)) to re-sample equal numbers of TAA and controls with repetitions. Such bootstrapping strategy takes into account the complex relationship between genes as well as the variability between samples; it also allows confidence estimation for classifier gene selection. To identify an optimal classifier gene set, we removed one gene at a time and estimated corresponding prediction accuracy using a 10-fold cross validation on the training set (Tibshirani et al., Proc Natl Acad Sci USA 99, 6567-72 (2002)). A minimal 41-gene set was selected based on its optimal consistency in predicting accuracy (average 78±6%), sensitivity (average 81±6%) and specificity (average 75±6%) (FIG. 3A, and FIG. 9) even though fewer genes can produce reasonably good results as well (FIG. 6). Principal component analysis using the 41 classifier genes can segregate TAA from control samples in three dimensional spaces with only a few exceptions (FIG. 3B).

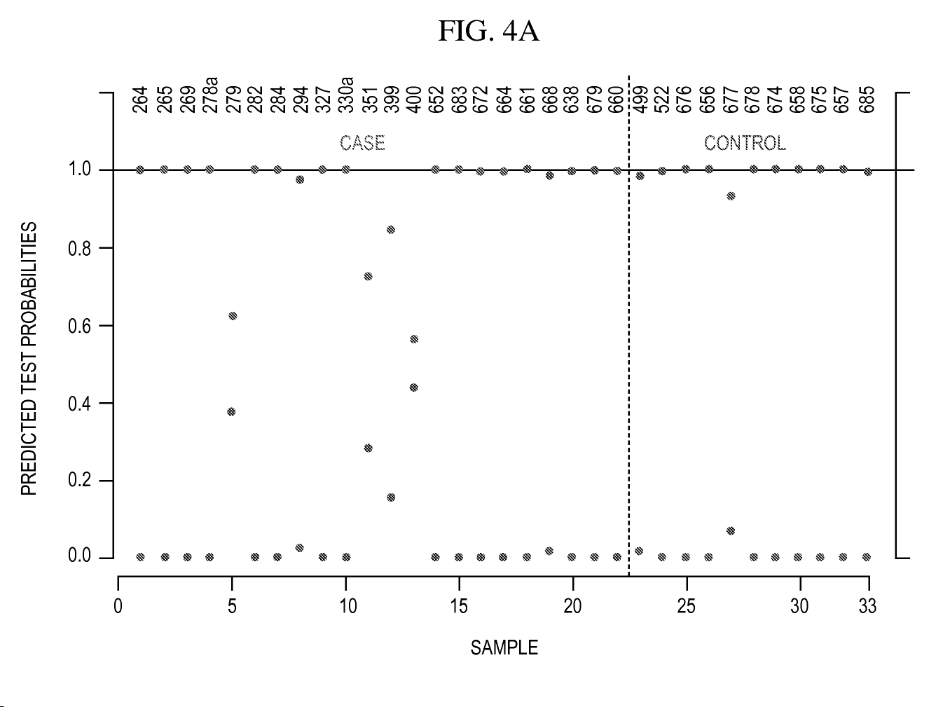

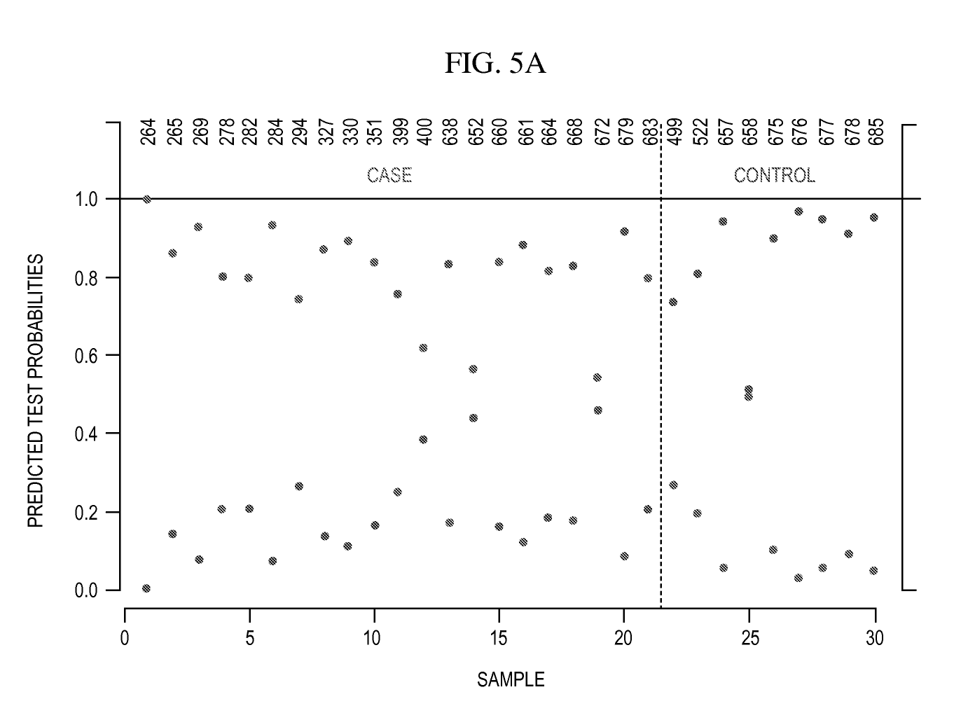

To further test the 41-gene prediction model, we generated an independent test set consisting of 33 peripheral blood samples (22 TAA and 11 controls) and their gene expression profiles were analyzed using Applied Biosystems Human Genome Arrays. Fifteen of these independent samples were collected and analyzed at the same time as the 61 training samples; the remaining 18 testing samples were collected at least one year later and their expression profiles were analyzed by different operators and microarray instruments. The predicting accuracy, sensitivity, and specificity for the testing set are 78%, 72%, and 90% respectively (FIG. 4), very similar to the results estimated by the 10-fold cross validation on the training set (FIG. 3A).

### TaqMan® Assay-Based Real Time PCR Validation

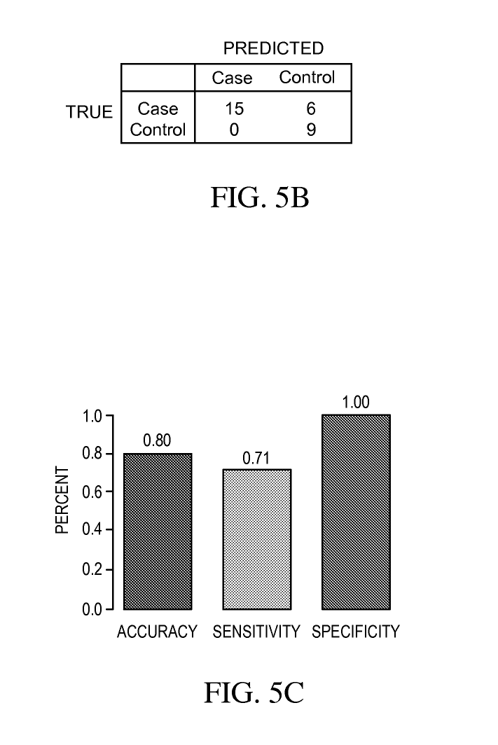

To further validate the TAA signature genes identified in this study, we performed real-time PCR validation using TaqMan® Gene Expression Assays on 82 samples from the original 94 samples (50 training samples, 32 testing samples, 12 samples were excluded because of insufficient amount of total RNA). 71 genes were chosen for this validation, including the 41 classifier genes that classify TAA vs. controls, plus a subset of candidate signature genes characterizing sub-types of TAA (18 for ascending vs. descending TAA and 10 for familial vs. sporadic TAA) and two endogenous controls that can be used for normalization between samples. Similar prediction performance was achieved using TaqMan data compared to that of microarray data: the predicting and accuracy, sensitivity and specificity for the testing set based on TaqMan data are 80%, 71%, and 100% respectively (FIG. 5). In addition to the 41 classifier genes distinguishing TAA vs. normal samples, 28 other signature genes characterizing sub-types of TAA were also validated by TaqMan real time PCR. Even though the average fold changes for most of these signature genes are relatively small (mostly <2-fold), the fold change correlation and direction between the TaqMan data and the microarray data are in good agreement (Table 1).

### ADDITIONAL DISCUSSION

Through a whole-genome gene expression profiling analysis in a relatively large number of thoracic aneurysm patients and controls, this investigation has successfully identified a distinct molecular signature in peripheral blood cells that distinguishes TAA patients from controls. Derived from microarray analysis and further validated by TaqMan realtime PCR, we have identified a set of 41 classifier genes that predict TAA disease with overall prediction accuracy of 78-80%. This accuracy level is promising for potential clinical application; therefore the set of 41 genes identified in this study provides diagnostic markers for TAA disease.

Furthermore, this investigation finds different RNA profiles for ascending and descending TAA, consistent with the current understanding that ascending and descending thoracic aortic aneurysms are very different diseases with widely differing embryology, pathophysiology, and clinical manifestations. Also, this investigation finds different profiles for patients with a familial pattern of aneurysm disease compared to those with sporadic aneurysm. This finding is reminiscent of increasing recognition that this disease is indeed transmitted in an inherited fashion in at least one-fifth of patients (Albornoz et al. Ann Thorac Surg 82, 1400-5 (2006)). While genetic tests may be beneficial for risk assessments of familial TAA, the identified gene expression signature unique to sporadic TAA hold potential in developing gene-expression based test for identifying individuals at risk for sporadic TAA without genetic imprints. Even though the expression signature identified in this study was derived from peripheral blood cells, surrogate samples instead of direct diseased tissues, the molecular pathways associated with the TAA signature genes may still shed light into the pathogenesis of aortic aneurysm disease, in particular in view of the intimate links between the aneurysm diseases, inflammation, and cellular immune responses.

Our finding of associated biological pathways are also consistent with the work of Taketani and colleagues (Int Heart J 46, 265-77 (2005)) who examined the expression profiles of surgically resected specimens from a relatively small number of TAA patients. For example, the present teachings reveal that the interleukin signaling pathway was significantly over-represented by the up-regulated TAA signature genes. Among this pathway, IL10, one of the characteristic TH2-derived cytokines, has been reported to tend to limit the cytotoxic potential of macrophages and to reduce the expression of proinflammatory mediators such as cytokines or matrix metalloproteinases (MMPs). In addition, study has shown that IL10 specifically over-expressed in abdominal aortic aneurysm tissue while absent in tissues derived from normal individuals or carotid atheroma patients (Schonbeck et al., Am J Pathol 161, 499-506 (2002)). High IL10 production in both PBMC and serum has also been associated with autoimmune diseases such as rheumatoid arthritis systemic lupus erythematosus (SLE) (Beebe et al., Cytokine Growth Factor Rev 13, 403-12 (2002)). While some evidence has suggested that the polymorphisms in IL-10 gene promoter may predispose to SLE (D'Alfonso et al., Genes Immun 1, 231-3 (2000); Mehrian et al. Arthritis Rheum 41, 596-602 (1998)), the molecular basis of increased production of IL-10 in peripheral blood of TAA patients remains to be further investigated. Another finding of our study is that mitogen-activated kinase protein (MAPK6 and MAPK7) are significantly over-expressed in TAA whole blood samples. Previous physiologic and histological analysis using mice model showed that MAPK7 is critical for endothelial function and maintenance of blood vessel integrity (Hayashi et al., J Clin Invest 113, 1138-48 (2004)). On the other hand, our study identified some of the significantly down-regulated genes in TAA peripheral blood that are associated with the Apoptosis/FAS signaling pathway.

Thus, the present teachings provide a method of diagnosing a human subject with TAA, the method comprising: detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with TAA. In some embodiments, the plurality of genes associated with TAA is the forty-one genes in FIG. 9. In some embodiments, the prediction accuracy is greater than 70 percent, 71 percent, 72 percent, 73 percent, 74 percent, 75 percent, 76 percent, 77 percent, 78 percent, 79 percent, 80 percent, 81 percent, 82 percent, 83 percent, 84 percent 85 percent, 90 percent, 95 percent, or 99 percent. In some embodiments, the detecting comprises a multiplexed PCR, followed by a plurality of lower-plex PCRs. Examples of multiplexed PCR, as well as multiplexed PCR followed by lower-plex PCRs, can be found for example in U.S. Pat. No. 6,605,451, and U.S. patent application Ser. No. 11/090,930. For example, the Applied Biosystems pre-amplification kit can be employed to amplify a collection of genes in a multiplexed PCR. Thereafter, lower-plex PCR can be performed, such as single-plex PCR where a given gene is quantitated with a TaqMan® 5′ nuclease detector probe. In some embodiments, the detecting comprises hybridization to an array. Exemplary array methods can be found described for example in U.S. Pat. No. 6,797,470, U.S. Pat. No. 7,108,969, and U.S. Pat. No. 6,905,826. In some embodiments, the method further comprises a surgical treatment. For example, after collecting the gene expression information from a test sample, it may be desirable to operate on TAA positive patients. Methods of performing surgical treatment can be found, for example, in Coady et al., J Thorac Cardiovasc Surg. 1997 March; 113(3):476-91; discussion 489-91; Morales et al., Ann Thorac Surg. 1998 November; 66(5):1679-83; Coady et al., Ann Thorac Surg. 1999 June; 67(6):1922-6; Elefteriades et al., Ann Thorac Surg. 1999 June; 67(6):2002-5; Coady et al., Cardiol Clin. 1999 November; 17(4):637-57; Rizzo et al., Cardiol Clin. 1999 November; 17(4):797-805; Coady et al., Cardiol Clin. 1999 November; 17(4):827-39; Tittle et al., J Thorac Cardiovasc Surg. 2002 June; 123(6):1051-9; Elefteriades et al., Ann Thorac Surg. 2002 November; 74(5):51877-80; Elefteriades, Adv Cardiol. 2004; 41:75-86. Review; Elefteriades, Sci Am. 2005 August; 293(2):64-71; Elefteriades et al., Ann Thorac Surg. 2005 September; 80(3):1098-100; Gallo A et al., Semin Thorac Cardiovasc Surg. 2005 Fall; 17(3):224-35; Davies et al., Ann Thorac Surg. 2006 January; 81(1):169-77; Dobrilovic et al., J Thorac Cardiovasc Surg. April; 131(4):777-8; Elefteriades et al., J Thorac Cardiovasc Surg. 2007 February; 133(2):285-8; Gega et al., Ann Thorac Surg. 2007 September; 84(3):759-66; discussion 766-7).

In some embodiments, the present teachings provide a method of distinguishing ascending thoracic aortic aneurysm from descending thoracic aortic aneurysm comprising; detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with an ascending aortic aneurysm, wherein the plurality of genes in the test sample are overexpressed in the ascending aortic aneurysm as compared with the control sample. In some embodiments, the plurality of genes associated with TAA is the one-hundred and forty-four genes in FIG. 7. In some embodiments, the detecting comprises a multiplexed PCR, followed by a plurality of lower-plex PCRs. In some embodiments, the detecting comprises hybridization to an array. In some embodiments, the method further comprises a surgical treatment.

In some embodiments, the present teachings provide a method of distinguishing sporadic thoracic aortic aneurysm from familial thoracic aortic aneurysm comprising; detecting a level of expression of a plurality of genes associated with TAA in a test sample from the human subject, wherein the test sample is blood; and, comparing the level of expression of a plurality of genes in the test sample with a level of expression of a plurality of genes in a control sample, wherein the level of expression of the plurality of genes in the test sample differs from the level of expression of the plurality of genes in the control sample when the thoracic aortic aneurysm is sporadic, wherein the plurality of genes in the test sample are overexpressed in the test sample as compared with the control sample. In some embodiments, the plurality of genes upregulated in the test sample is the 113 genes in FIG. 8. In some embodiments, the detecting comprises a multiplexed PCR, followed by a plurality of lower-plex PCRs. In some embodiments, the detecting comprises hybridization to an array. In some embodiments, the method further comprises a surgical treatment.

In some embodiments, the methods of the present teachings comprise querying the expression of the genes provided in FIGS. 7, 8, and/or 9. In some embodiments, the methods of the present teachings consist essentially of querying the expression of the genes provided in FIGS. 7, 8, and/or 9. In some embodiments, the methods of the present teachings consist of querying the expression of the genes provided in FIG. 7, 8, or 9.

The present teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

Thus, in some embodiments the present teachings provide kits for monitoring TAA gene expression, for example kits comprising PCR primer pairs, and optionally a detector probe, such as a 5′ nuclease probe. In some embodiments, such PCR kits can further comprise a master mix, a polymerase, various buffers, nucleotides, and appropriate reaction vessels.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method of diagnosing a human subject with TAA, the method comprising;
detecting a level of expression of a plurality of genes associated with TAA in a test sample from the human subject, wherein the test sample is blood; and,
comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with TAA.

2. The method according to claim 1 wherein the plurality of genes associated with TAA is the forty-one genes in FIG. 9.

3. The method according to claim 1 wherein the prediction accuracy is greater than 70 percent.

4. The method according to claim 1 wherein the detecting comprises a multiplexed PCR, followed by a plurality of lower-plex PCRs.

5. The method according to claim 1 wherein the detecting comprises hybridization to an array.

6. The method according to claim 1 further comprising a surgical treatment.

7. A method of distinguishing ascending thoracic aortic aneurysm from descending thoracic aortic aneurysm comprising;
detecting a level of expression of a plurality of genes associated with TAA in a test sample from the human subject, wherein the test sample is blood; and,
comparing the level of expression of a plurality of genes in the test sample with a level of expression of a plurality of genes in a control sample, wherein the level of expression of the plurality of genes in the test sample differs from the level of expression of the plurality of genes in the control sample when the subject is afflicted with an ascending aortic aneurysm, wherein the plurality of genes in the test sample are overexpressed in the ascending aortic aneurysm as compared with the control sample.

8. The method according to claim 7 wherein the plurality of genes associated with TAA is the one-hundred and forty-four genes in FIG. 9.

9. The method according to claim 7 wherein the detecting comprises a multiplexed PCR, followed by a plurality of lower-plex PCRs.

10. The method according to claim 7 wherein the detecting comprises hybridization to an array.

11. The method according to claim 7 further comprising a surgical treatment.

12. A method of distinguishing sporadic thoracic aortic aneurysm from familial thoracic aortic aneurysm comprising;
detecting a level of expression of a plurality of genes associated with TAA in a test sample from the human subject, wherein the test sample is blood; and,
comparing the level of expression of a plurality of genes in the test sample with a level of expression of a plurality of genes in a control sample, wherein the level of expression of the plurality of genes in the test sample differs from the level of expression of the plurality of genes in the control sample when the thoracic aortic aneurysm is sporadic, wherein the plurality of genes in the test sample are overexpressed in the test sample as compared with the control sample.

13. The method according to claim 12 wherein the plurality of genes upregulated in the test sample is the 113 genes in FIG. 8.

14. The method according to claim 12 wherein the detecting comprises a multiplexed PCR, followed by a plurality of lower-plex PCRs.

15. The method according to claim 12 wherein the detecting comprises hybridization to an array.

16. The method according to claim 14 further comprising a surgical treatment.
